(19) United States
(12) Patent Application Publication
(10) Pub. No.: US 2021/0371455 A1
(43) Pub. Date: Dec. 2, 2021

(54) METHODS AND COMPOSITIONS COMPRISING REDUCED LEVEL OF HOST CELL PROTEINS

(71) Applicant: Regeneron Pharmaceuticals, Inc., Tarrytown, NY (US)

(72) Inventors: Sisi Zhang, White Plains, NY (US); Hui Xiao, Scarsdale, NY (US)

(21) Appl. No.: 17/340,763

(22) Filed: Jun. 7, 2021

Related U.S. Application Data
(63) Continuation-in-part of application No. 16/925,664, filed on Jul. 10, 2020.
(60) Provisional application No. 62/872,515, filed on Jul. 10, 2019.
(60) Provisional application No. 62/979,835, filed on Feb. 21, 2020.

(51) Int. Cl. C07K 1/22; G01N 30/72

(52) U.S. Cl. CPC C07K 1/22; G01N 2333/914; G01N 30/72

(57) ABSTRACT

The present disclosure pertains to compositions with reduced presence of host-cell proteins and methods of making such compositions. In particular, it pertains to compositions methods of making compositions with reduced presence of host-cell proteins from a host-cell.

### CROSS-REFERENCE TO RELATED APPLICATIONS

This application is related to U.S. patent application Ser. No. 16/925,664, filed Jul. 10, 2020, which claims priority to and the benefit of U.S. Provisional Patent Application No. 62/979,835, filed Feb. 21, 2020 and U.S. Provisional Patent Application No. 62/872,515, filed Jul. 10, 2019 which are each herein incorporated by reference.

### SEQUENCE LISTING

The instant application contains a Sequence Listing which has been submitted electronically in ASCII format and is hereby incorporated by reference in its entirety. Said ASCII copy, created on Sep. 22, 2020, is name 070816-01322)SL.txt and is 12,183 Bytes.

### FIELD

The present invention generally pertains to compositions with reduced presence of host-cell proteins and methods of making such compositions. In particular, the present invention generally pertains to compositions and methods of making compositions with reduced presence of host-cell proteins from a host-cell.

### BACKGROUND

Among drug products, protein-based biotherapeutics are an important class of drugs that offer a high level of selectivity, potency and efficacy, as evidenced by the considerable increase in clinical trials with monoclonal antibodies (mAbs) over the past several years. Bringing a protein-based biotherapeutic to the clinic can be a multiyear undertaking requiring coordinated efforts throughout various research and development disciplines, including discovery, process and formulation development, analytical characterization, and pre-clinical toxicology and pharmacology.

One critical aspect for a clinically and commercially viable biotherapeutic is stability of the drug product in terms of the manufacturing process as well as shelf-life. This often necessitates appropriate steps to help increase physical and chemical stability of the protein-based biotherapeutics throughout the different solution conditions and environments necessary for manufacturing and storage with minimal impact on product quality, including identifying molecules with greater inherent stability, protein engineering, and formulation development. Surfactants, such as, polysorbate are often used to enhance the physical stability of a protein-based biotherapeutic product. Over seventy percent of marketed monoclonal antibody therapeutics contain between 0.001% and 0.1% polysorbate, a type of surfactant, to impart physical stability to the protein-based biotherapeutics. Polysorbates are susceptible to auto-oxidation and hydrolysis, which results in free fatty acids and subsequent fatty acid particle formation. The degradation of polysorbate can adversely affect the drug product quality since polysorbate can protect against interfacial stress, such as aggregation and adsorption. Presence of host cell proteins (HCPs) can be a likely cause of degradation of polysorbates in a formulation. In addition to affecting the polysorbates, HCP impurities present at low levels can further cause an immunogenic reaction. Thus, host cell proteins in drug products need to be monitored.

Analytical methods for assays used to characterize HCPs should display sufficient accuracy and resolution. Direct analysis of HCPs can require isolation of the product in a sufficiently large amount for the assay, which is undesirable and has only been possible in selected cases. Hence, it is a challenging task to determine the workflow and analytical tests required to characterize HCPs in a sample.

It will be appreciated that a need exists for compositions with reduced level of host-cell proteins that can degrade polysorbate, and methods for preparing such compositions as well as one or more methods to detect those proteins.

### SUMMARY

Maintaining stability of drug formulations, not only during storage, but also during manufacturing, shipment, handling and administration, is a significant challenge. Among drug products, protein biotherapeutics are gaining popularity due to their success and versatility. One of the major challenges for protein biotherapeutics development is to overcome the limited stability of the proteins which can be affected by the presence of host-cell protein. Evaluation of its effect on the drug formulation and reduction of such host-cell proteins can be an important step in the drug formulation development, followed by methods to prepare the drug formulation so as to have reduced host-cell proteins and increased stability owing to the reduced host-cell proteins.

In one exemplary embodiment, the composition can comprise a protein of interest purified from mammalian cells and a residual amount of sialate o-acetylesterase. In one aspect, the residual amount of sialate o-acetylesterase (SIAE) is less than about 5 ppm. In another aspect, the composition can further comprise a surfactant. In yet a further aspect, the surfactant can be a hydrophilic nonionic surfactant. In another aspect, the surfactant can be a sorbitan fatty acid ester. In a specific aspect, the surfactant can be a polysorbate. In another specific aspect, the concentration of the polysorbate in the composition can be about 0.01% w/v to about 0.2% w/v. In a further specific aspect, the surfactant can be a polysorbate 20. In one aspect, the mammalian cells can include a CHO cell. In one aspect, the mammalian cell can include SIAE-knockout cell. In a specific aspect, the mammalian cells can include a SIAE-knockout CHO cell. In one aspect, the SIAE can be CHO-SIAE.

In one aspect, the sialate o-acetylesterase protein can cause degradation of polysorbate 20. In another aspect, the sialate o-acetylesterase can be cytosolic sialic acid esterase isoform. In yet another aspect, the sialate o-acetylesterase can be lysosomal sialic acid esterase isoform.

In one aspect, the composition can be a parenteral formulation.

In one aspect, the protein of interest can be a monoclonal antibody, a polyclonal antibody, a bispecific antibody, an antibody fragment, a fusion protein, or an antibody-drug complex. In one aspect, the concentration of the protein of interest can be about 20 mg/mL to about 400 mg/mL.

In one aspect, the composition can further comprise one or more pharmaceutically acceptable excipients. In another aspect, the composition can further comprise a buffer selected from a group consisting of histidine buffer, citrate buffer, alginate buffer, and arginine buffer. In one aspect, the composition can further comprise a tonicity modifier. In yet another aspect, the composition can further comprise sodium phosphate.

In one exemplary embodiment, the composition can comprise a protein of interest purified from mammalian cells and a residual amount of lysosomal acid lipase. In one aspect, the residual amount of lysosomal acid lipase is less than about 1 ppm. In another aspect, the composition can further comprise a surfactant. In one specific aspect, the surfactant can be a hydrophilic nonionic surfactant. In another specific aspect, the surfactant can be a sorbitan fatty acid ester. In yet another specific aspect, the surfactant can be a polysorbate. In a specific aspect, the concentration of the polysorbate in the composition can be about 0.01% w/v to about 0.2% w/v. In a further specific aspect, the surfactant can be polysorbate 20 and polysorbate 80. In one aspect, the mammalian cells can include a CHO cell.

In one aspect, the mammalian cell can include a LIPA-knockout cell. In a specific aspect, the mammalian cell can include a LIPA-knockout CHO cell.

In one aspect, the lysosomal acid lipase can cause degradation of the polysorbate. In one aspect, the lysosomal acid lipase can be CHO-lysosomal acid lipase.

In one aspect, the composition can be a parenteral formulation.

In one aspect, the protein of interest can be a monoclonal antibody, a polyclonal antibody, a bispecific antibody, a fusion protein, an antibody fragment or an antibody-drug complex.

In one aspect, the composition can further comprise one or more pharmaceutically acceptable excipients. In one aspect, the composition can further comprise a buffer selected from a group consisting of histidine buffer, citrate buffer, alginate buffer, and arginine buffer. In one aspect, the composition can further comprise a tonicity modifier. In one aspect, the composition can further comprise sodium phosphate. In one aspect, the concentration of the protein of interest can be about 20 mg/mL to about 400 mg/mL.

The disclosure, at least in part, provides a method of preparing a composition having a protein of interest and less than about 5 ppm of sialate o-acetylesterase and/or less than about 1 ppm of lysosomal acid lipase.

In one exemplary embodiment, the method of preparing a composition having a protein of interest and less than about 5 ppm of sialate o-acetylesterase and/or less than about 1 ppm of lysosomal acid lipase can comprise (a) culturing mammalian cells to produce the protein of interest to form a sample matrix; (b) contacting the sample matrix to a first chromatography resin; and (c) washing the bound protein of interest to form an eluate.

In one aspect, the method of preparing a composition can further comprise step (d) contacting the eluate obtained from step (c) to a second chromatography resin. In another aspect, the method of preparing a composition can further comprise step (e) collecting a flow-through from washing the second chromatography resin. In a further aspect, the method of preparing a composition can further comprise step (f) contacting the flow-through to a third chromatography resin. In a yet further aspect, the method of preparing a composition can further comprise step (g) collecting a second flow-through from washing the third chromatography resin.

In one aspect, the method of preparing a composition can further comprise a step of filtering the eluate. In one aspect, the method of preparing a composition can further comprise a step of filtering the flow-through. In another aspect, the method of preparing a composition can further comprise a step of filtering the second flow-through. In one aspect, the filtration can be carried out using viral filtration. In another aspect, the filtration can be carried out using UF/DF. In one aspect, the first chromatographic resin can be protein A chromatographic resin, anion-exchange chromatographic resin, cation-exchange chromatographic resin, mixed-mode chromatographic resin or hydrophobic interaction chromatographic resin. In a specific aspect, the first chromatographic resin can be protein A chromatographic resin. In one aspect, the second chromatographic resin can be selected from protein A chromatographic resin, anion-exchange chromatographic resin, cation-exchange chromatographic resin, mixed-mode chromatographic resin or hydrophobic interaction chromatographic resin. In a specific aspect, the first chromatographic resin can be an ion-exchange chromatographic resin. In a specific aspect, the first chromatographic resin can be an anion-exchange chromatographic resin. In one aspect, the third chromatographic resin can be protein A chromatographic resin, anion-exchange chromatographic resin, cation-exchange chromatographic resin or hydrophobic interaction chromatographic resin. In a specific aspect, the first chromatographic resin can be a hydrophobic interaction chromatographic resin.

In one aspect, the method of preparing a composition can further comprise a purification step using beads having anti-sialate o-acetylesterase antibody. In a specific aspect, the purification step can be carried out by contacting to the beads one or more of the following: the sample matrix, the eluate, the flow-through or the second flow-through. In one aspect, the anti-sialate o-acetylesterase antibody can be of human origin. In another aspect, the anti-sialate o-acetylesterase antibody can be of hamster origin.

In one aspect, the method of preparing a composition can further comprise a purification step using beads having anti-lysosomal acid lipase antibody. In a specific aspect, the purification step can be carried out by contacting to the beads one or more of the following: the sample matrix, the eluate, the flow-through or the second flow-through. In one aspect, the anti-lysosomal acid lipase antibody can be of human origin. In another aspect, the anti-lysosomal acid lipase antibody can be of hamster origin.

In one aspect, the composition has less than about 5 ppm of sialate o-acetylesterase. In another aspect, the composition has less than about 1 ppm of lysosomal acid lipase. In yet another aspect, the composition has less than about 5 ppm of sialate o-acetylesterase and less than about 1 ppm of lysosomal acid lipase.

In one exemplary embodiment, the disclosure provides a method of depleting sialate o-acetylesterase levels in a sample matrix. In one aspect, the method of depleting sialate o-acetylesterase levels in a sample matrix can comprise contacting the sample matrix having sialate o-acetylesterase to a resin having anti-sialate o-acetylesterase antibody. In one aspect, the method can further comprise washing the resin with a wash buffer. In another aspect, the method can further comprise collecting wash fractions from the washing the resin. In one aspect, the wash fractions can have a reduced concentration of sialate o-acetylesterase than sialate o-acetylesterase in the sample matrix. In one aspect, the sample matrix can comprise polysorbate. In one aspect, the resin can be a magnetic bead. In one aspect, the amount of anti-sialate o-acetylesterase antibody to the resin can be about 1 μg/g to about 50 μg/g. In one aspect, the anti-sialate o-acetylesterase antibody can be of human origin. In one aspect, the anti-sialate o-acetylesterase antibody can be of hamster origin. In one aspect, the amount of sialate o-acetylesterase in the wash fractions can be at least about two-fold reduced compared to the amount of sialate o-acetylesterase in the sample matrix.

In one exemplary embodiment, the disclosure provides a method of depleting lysosomal acid lipase levels in a sample matrix. In one aspect, the method of depleting lysosomal acid lipase levels in a sample matrix can comprise contacting the sample matrix having lysosomal acid lipase to a resin having anti-lysosomal acid lipase antibody. In one aspect, the method can further comprise washing the resin with a wash buffer. In yet another aspect, the method can further comprise collecting wash fractions from the washing. In one aspect, the wash fractions can have a reduced concentration of lysosomal acid lipase than lysosomal acid lipase in the sample matrix. In one aspect, the sample matrix can comprise polysorbate. In one aspect, the resin can be a magnetic bead. In one aspect, the amount of anti-lysosomal acid lipase antibody to the resin can be about 1 μg/g to about 50 μg/g. In one aspect, the anti-lysosomal acid lipase can be of human origin. In one aspect, the anti-lysosomal acid lipase antibody can be of hamster origin. In one aspect, the amount of lysosomal acid lipase in the wash fractions can be at least about two-fold reduced compared to the amount of lysosomal acid lipase in the sample matrix.

In one exemplary embodiment, the disclosure provides a method of detecting sialate o-acetylesterase in a sample matrix. In one aspect, the method of detecting sialate o-acetylesterase in a sample matrix can comprise contacting the sample matrix with a resin having a biotinylated anti-sialate o-acetylesterase antibody. In one aspect, the method can further comprise incubating the sample matrix with the resin. In another aspect, the method can further comprise performing elution on the resin of to form an eluate. In one aspect, the resin can be a magnetic bead. In one aspect, the elution can be performed using one or more solvents selected from acetonitrile, water and acetic acid.

In one aspect, the method can further comprise adding hydrolyzing agent to the eluate to obtain digests. In a specific aspect, the hydrolyzing agent can be trypsin. In one aspect, the method can further comprise analyzing the digests to detect the sialate o-acetylesterase. In one aspect, the digests can be analyzed using a mass spectrometer. In a specific aspect, the mass spectrometer can be a tandem mass spectrometer. In another specific aspect, the mass spectrometer can be coupled to a liquid chromatography system. In yet another specific aspect, the mass spectrometer can be coupled to a liquid chromatography-multiple reaction monitoring system.

In one aspect, the method can further comprise adding protein denaturing agent to the eluate. In a specific aspect, the protein denaturing agent can be urea. In one aspect, the method can further comprise adding protein reducing agent to the eluate. In a specific aspect, the protein reducing agent can be DTT (dithiothreitol). In one aspect, the method can further comprise adding protein alkylating agent to the eluate. In a specific aspect, the protein alkylating agent can be iodoacetamide.

In one exemplary embodiment, the disclosure provides a method of detecting lysosomal acid lipase in a sample matrix. In one aspect, the method of detecting lysosomal acid lipase in a sample matrix can comprise contacting the sample matrix with a resin having a biotinylated anti-lysosomal acid lipase antibody. In one aspect, the method can further comprise incubating the sample matrix with the resin. In one aspect, the method can further comprise performing elution on the resin of to form an eluate. In one aspect, the resin can be a magnetic bead. In one aspect, the elution can be performed using one or more solvents selected from acetonitrile, water and acetic acid.

In one aspect, the method can further comprise adding hydrolyzing agent to the eluate to obtain digests. In a specific aspect, the hydrolyzing agent can be trypsin. In one aspect, the method can further comprise analyzing the digests to detect the lysosomal acid lipase. In one aspect, the digests can be analyzed using a mass spectrometer. In a specific aspect, the mass spectrometer can be a tandem mass spectrometer. In another specific aspect, the mass spectrometer can be coupled to a liquid chromatography system. In yet another specific aspect, the mass spectrometer can be coupled to a liquid chromatography-multiple reaction monitoring system.

In one aspect, the method can further comprise adding protein denaturing agent to the eluate. In a specific aspect, the protein denaturing agent can be urea. In one aspect, the method can further comprise adding protein reducing agent to the eluate. In a specific aspect, the protein reducing agent can be DTT. In one aspect, the method can further comprise adding protein alkylating agent to the eluate. In a specific aspect, the protein alkylating agent can be iodoacetamide.

These, and other, aspects of the invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows the protein sequence alignment of human SIAE (SEQ ID NO.: 13) and CHO SIAE (SEQ ID NO.: 12).

FIG. 2 shows as schematic diagram of the SIAE depletion experiment according to one exemplary embodiment, wherein Dynabeads magnetic beads are covalently coupled with Anti-SIAE monoclonal antibody and used for immunoprecipitating (IP), the original mAb (A) and flow through (B) were incubated with 0.1% PS20 at 45° C. for 5 days and subjected to PS20 degradation measurement and a non-relevant antibody served as the negative control by replacing anti-SIAE monoclonal antibody (C).

FIG. 3 shows the chemical structure of major expected polyols esters (POE esters) in polysorbates according to an exemplary embodiment, wherein the polysorbates are mainly composed of fatty acid esters shared common sorbitan or isosorbide head group, wherein lauric acid is the main fatty acid for PS20 and oleic acid is the main fatty acid for PS80.

FIG. 4A shows a chart obtained on separation and detection of PS20 standard (A) and PS20 in mAb formulation (B) by online coupling 2D-LC with CAD according to an exemplary embodiment, with major peaks labeled as POE sorbitan monolaurate (1), POE isosorbide monolaurate (2), POE sorbitan monomyristate (3), POE isosorbide monomyristate (4), POE isosorbide monopalmitate (5), POE isosorbide monosterate (6), POE sorbitan mixed diesters (7-9), POE sorbitan trilaurate and POE sorbitan tetralaurate (10).

FIG. 4B shows a chart obtained on separation and detection of PS80 standard (A) and PS80 in mAb formulation (B) by online coupling 2D-LC with CAD according to an exemplary embodiment, with major peaks labeled as POE isosorbide monolinoleate (1), POS sorbitan monooleate (2), POE isosorbide monooleate and POE monooleate (3), POE sorbitan di-oleate (4), POE isorbide di-oleate (5), and POE sorbitan mixed trioleate and tetraoleate (6).

FIG. 5A shows the representative total ion current (TIC) profile of PS20 according to an exemplary embodiment, with major peaks labeled as POE sorbitan monolaurate (1), POE isosorbide monolaurate (2), POE sorbitan monomyristate (3), POE isosorbide monomyristate (4), POE isosorbide monopalmitate (5), POE isosorbide monosterate (6), POE sorbitan mixed diesters (7-9), POE sorbitan trilaurate and POE sorbitan tetralaurate (10).

FIG. 5B shows the representative total ion current (TIC) profile of PS80 according to an exemplary embodiment, with major peaks labeled as POE isosorbide monolinoleate (1), POS sorbitan monooleate (2), POE isosorbide monooleate and POE monooleate (3), POE sorbitan di-oleate (4), POE isorbide di-oleate (5), and POE sorbitan mixed trioleate and tetraoleate (6).

FIG. 6 depicts a chromatogram of 0.1% PS20 solution incubated with 1 ppm (I), 2.5 ppm (II), 10 ppm (III) recombinant sialate O-acetylesterase @45° C. in 10 mM Histidine, pH 6 for 0 day (A, T0), and 10 days (B, T10) according to an exemplary embodiment.

FIG. 7 shows chromatograms for (i) 0.1% PS20 solution incubated with 5 ppm recombinant sialate O-acetylesterase @45° C. in 10 mM Histidine, pH 6 for 0 day (A, T0), and 5 days (B, T5) (upper panel) and (ii) 0.1% PS20 in 75 mg/mL mAb incubated @45° C. in 10 mM Histidine, pH 6 for 0 day (C, T0), and 5 days (D, T5) (lower panel) according to an exemplary embodiment.

FIG. 8 shows the effects of pH on the PS20 degradation according to an exemplary embodiment, wherein the upper panel shows comparison of 0.1% PS20 degradation incubated with recombinant SIAE at pH 6.0 (A) and pH 8.0 (B); and 0.1% PS20 degradation incubated with 75 mg/mL mAb-1 at pH 6.0 (C) and pH 8.0 (D) and bottom panel shows comparison of 0.2% PS20 degradation incubated with recombinant SIAE at pH 6.0 (E) and pH 5.3 (F); and 0.2% PS20 degradation incubated with 150 mg/mL mAb-1 at pH 6.0 (G) and pH 5.3 (H).

FIG. 9 shows a calibration curve of two selected peptides LLSLTYDQK (SEQ ID NO.: 1) (filled square) and ELAVAAAYQSVR (SEQ ID NO.: 2) (filled circle) from recombinant sialate O-acetylesterase with mAb as matrix according to an exemplary embodiment.

FIG. 10 shows a correlation curve between remaining PS20 percentage and SIAE concentration according to an exemplary embodiment, wherein the SIAE concentration was quantitated by IP-MRM-MS using a calibration curve, the percentage of PS20 remaining was determined by using LC-CAD after 0.1% PS20 was incubated with various mAbs (filled circles, 75 mg/mL) at 45° C. for 5 days and filled square markers represent the in-process mAb-3 in four consecutive processing steps, which are Protein A, AEX, HIC and VF pool, respectively.

FIG. 11 shows a western blot of recombinant SIAE (I) according to an exemplary embodiment, where lanes 1, 2, 3 are pure SIAE loaded at an amount of 10 ng, 50 ng and 100 ng, respectively; lanes 4, 5, 6 are SIAE mixed with 100 μg mAb loaded at an amount of 10 ng, 50 ng and 100 ng, respectively and lane 7 is 100 μg mAb alone.

FIG. 12 shows the percentage of PS20 remaining in original mAb, SIAE-depleted mAb and negative control against incubation time for mAb-4, where the original mAb, SIAE-depleted mAb and negative control are indicated by filled circle with solid line, filled diamond with dashed line and filled triangle with dashed line according to an exemplary embodiment.

FIG. 13 shows the percentage of PS20 remaining in original mAb, SIAE-depleted mAb and negative control against incubation time for mAb-5, where the original mAb, SIAE-depleted mAb and negative control are indicated by filled circle with solid line, filled diamond with dashed line and filled triangle with dashed line according to an exemplary embodiment.

FIG. 14 shows a plot of the remaining SIAE concentration of mAb-5 after SIAE depletion (filled diamond) was measured by IP-MRM-MS and plotted together with other mAbs measured according to an exemplary embodiment.

FIG. 15 shows a chromatogram of a solution of 0.1% PS80 incubated with spiked-in recombinant sialate O-acetylesterase (50 ppm) @45° C. in 10 mM Histidine, pH 6 for 0 day (A, T0), and 5 days (B, T5) according to an exemplary embodiment.

FIG. 16 shows the representative CAD profile of PS20 in a formulation with mAb-4 with major peaks labeled, containing sorbitan monoester, isosorbide monoester and diesters with a variety of fatty acid chains according to an exemplary embodiment.

FIG. 17 shows a CAD profile of 0.2% PS20 incubated with 10 ppm LAL and 10 ppm SIAE according to an exemplary embodiment.

FIG. 18 shows the percentage of PS20 remaining in original mAb preparation and LAL-depleted mAb preparation plotted against incubation time in days according to an exemplary embodiment.

FIG. 19 shows the percentage of PS20 remaining in mAb-1 formulation, wherein mAb-1 was prepared from LIPA-knockout CHO cell line and a control CHO cell line plotted against incubation time in days according to an exemplary embodiment.

FIG. 20 shows a chromatogram of PS80 degradation when incubated without LAL and with LAL at a concentration of 10 ppm and 20 ppm in 5 days according to an exemplary embodiment.

FIG. 21 shows a chromatogram of PS80 degradation when incubated with formulated mAb-1 obtained from different programs according to an exemplary embodiment.

FIG. 22 shows % PS 80 remaining for mAb-1 formulations with 0.1% PS80, wherein mAb-1 was prepared from LIPA-knockout CHO cell line and a control CHO cell line plotted against incubation time in days according to an exemplary embodiment.

FIG. 23 shows a western blot of PLBD2, Lane 1 is the molecular weight standard, Lane 2 is 40 ug mAb-8 containing PLBD2, lane 4 is 10 ng PLBD2 purchased from Origene, lane 5 is 10 ng CHO PLBD2 tagged with mmHis tag.

FIG. 24A shows a chromatogram of 0.1% PS20 in 200 μg/mL commercial PLBD2 spiked in 150 mg/mL mAb incubated @45° C. in 10 mM Histidine, pH 6 for for 0 day (A, T0), and 5 days (B, T5) according to an exemplary embodiment.

FIG. 24B shows a chromatogram of 0.1% PS20 in 200 μg/mL CHO PLBD2 spiked in 150 mg/mL mAb incubated @45° C. in 10 mM Histidine, pH 6 for for 0 day (A, T0), and 5 days (B, T5) according to an exemplary embodiment.

FIG. 24C shows a chromatogram of 0.1% PS80 in 200 μg/mL commercial PLBD2 spiked in 150 mg/mL mAb incubated @45° C. in 10 mM Histidine, pH 6 for for 0 day (C, T0), and 5 days (D, T5) according to an exemplary embodiment.

FIG. 24D shows a chromatogram of 0.1% PS80 in 200 μg/mL CHO PLBD2 spiked in 150 mg/mL mAb incubated @45° C. in 10 mM Histidine, pH 6 for for 0 day (C, T0), and 5 days (D, T5) according to an exemplary embodiment.

FIG. 25A shows a chromatogram of 0.1% PS20 in 75 mg/mL mAb-9 (generated by PLBD2 knockout cell line) incubated @45° C. in 10 mM Histidine, pH 6 for 0 day (A, T0), and 5 days (B, T5). % of PS20 degradation=(Peak Area (27.5-33 min) @T5)/(Peak Area (27.5-33 min) @T0) (upper panel) and a chromatogram of 0.1% PS80 in 100 mg/mL mAb incubated @45° C. in 10 mM Histidine, pH 6 for 0 day (C, T0), and 5 days (D, T5) (lower panel) according to an exemplary embodiment. % of PS80 degradation=(Peak Area (30-35 min) @T5)/(Peak Area (30-35 min)@T0).

FIG. 25B shows that PLBD2 knockout showed similar or higher lipase activity for PS20 and PS80 as control cell line as seen on comparison of 0.2% PS20 degradation incubated with 150 mg/mL mAb-8 generated from control cell line at pH 6.0 (A) and 150 mg/mL mAb-8 generated from PLBD2 knockout cell line at pH 6.0 (B) (top panel) and comparison of 0.1% PS80 degradation incubated with 75 mg/mL mAb-8 generated from control cell line at pH 6.0 (C) and 75 mg/mL mAb-9 generated from PLBD2 knockout cell line at pH 6.0 (D) (bottom panel) according to an exemplary embodiment.

FIG. 25C shows a western blot of PLBD2 in mAb-8 and mAb-9 PLBD2 knockout cell line. Lane 2 is 40 ug mAb-8 and lane 3 is 40 ug mAb-9 generated by PLBD2 knockout cell line according to an exemplary embodiment.

FIG. 26 shows a schematic diagram of the PLBD2 depletion experiment accordingly to an exemplary embodiment. Dynabeads magnetic beads were covalently coupled with Anti-PLBD2 monoclonal antibody and used for immunoprecipitating (IP). The original mAb (A) and flow through (B) were incubated with 0.1% PS at 45° C. for 5 days and subject to PS degradation measurement. A non-relevant antibody was served as the negative control by replacing anti-PLBD2 monoclonal antibody (C).

FIG. 27A shows a western blot of PLBD2, Lane 1 is MW standard, Lane 2 is 40 ug mAb-8 alone, lane 3 is 40 ug mAb-8 with PLBD2 being depleted completely, lane 4 is 40 ug mAb-8 with PLBD2 being partially depleted and lane 5 is 40 ug mAb-10 containing no PLBD2, carried out according to an exemplary embodiment.

FIG. 27B shows the percentage of PS20 remaining in original mAb-8, PLBD2-completely depleted mAb-8 and PLBD2 partially depleted mAb-8 plotted against incubation time. The original mAb, PLBD2 completely-depleted mAb and PLBD2 partially depleted mAb are indicated by filled circle with solid line, filled diamond with dashed line and filled triangle with dotted line.

FIG. 27C shows the percentage of PS80 remaining in original mAb-8, PLBD2-completely depleted mAb-8 and PLBD2 partially depleted mAb-8 plotted against incubation time. The original mAb, PLBD2 completely-depleted mAb and PLBD2 partially depleted mAb are indicated by filled circle with solid line, filled diamond with dashed line and filled triangle with dotted line.

FIG. 28 shows a calibration curve of two selected peptides YQLQFR (SEQ ID NO.: 3) (filled square) and SVLLDAASGQLR (SEQ ID NO.: 4) (filled circle) from recombinant CHO PLBD2 with mAb-10 as matrix.

FIG. 29 shows a correlation curve between remaining PS20 percentage and PLBD2 concentration. PLBD2 concentration were quantitated by MRM-MS using the calibration curve (SVLLDAASGQLR (SEQ ID NO.: 4)). The percentage of PS20 remaining was determined by using LC-CAD after 0.1% PS20 was incubated with various mAbs (filled circles, 75 mg/mL) at 45° C. for 5 days.

FIG. 30 shows the PS20 degradation in mAb-1 and mAb10-18 formulations comprising PS-20 at days 12 and 28 according to an exemplary embodiment.

FIG. 31 shows a chart of remaining % PS20 for relative LPL concentration to the mAb (ppm) for mAb-1 and mAb10-18 formulations comprising PS-20 at days 12 and 28 according to an exemplary embodiment.

FIG. 32 shows a chart of remaining % PS20 for relative LAL concentration to the mAb (ppm) for mAb-1 and mAb10-18 formulations comprising PS-20 at days 12 and 28 according to an exemplary embodiment.

FIG. 33 shows a chromatogram of samples incubated with 0.1% PS20 and 2 μg/mL mAb-1 @5° C. according to an exemplary embodiment.

FIG. 34 shows a chromatogram of samples from mAb-1 formulation comprising PS-20 incubated @5° C. according to an exemplary embodiment.

FIG. 35 shows a chart of remaining % PS20 for an incubated sample comprising 0.1% PS20 and 2 μg/mL and an incubated sample comprising PS20, LAL and 0.1 M Lalistat-2 at days 8 and 15 according to an exemplary embodiment.

FIG. 36 shows a chart of remaining % PS20 for an incubated sample comprising 0.1% PS20 and 2 μg/mL and an incubated sample comprising PS20, 2 LPL and 0.1 M Lalistat-2 at days 8 and 15 according to an exemplary embodiment.

FIG. 37 shows a chart of remaining % PS20 for mAb-1 formulation comprising 0.05% PS-20 incubated with and without 0.1 M Lalistat-2 at days 8 and 15 according to an exemplary embodiment.

### DETAILED DESCRIPTION

Host cell proteins (HCPs) are a class of impurities that should be removed from all cell-derived protein therapeutics. The FDA does not specify a maximum acceptable level of HCP, but HCP concentrations in final drug product should be controlled and reproducible from batch to batch (FDA, 1999). A primary safety concern relates to the possibility that HCPs can cause antigenic effects in human patients (Satish Kumar Singh, Impact of Product-Related Factors on Immunogenicity of Biotherapeutics, and 100 JOURNALS OF PHARMACEUTICAL SCIENCES 354-387 (2011)). In addition to adverse health consequences for the patient, enzymatically-active HCPs can potentially impact product quality during processing or long-term storage (Sharon X. Gao et al., Fragmentation of a highly purified monoclonal antibody attributed to residual CHO cell protease activity, 108 BIOTECHNOLOGY AND BIOENGINEERING 977-982 (2010); Flavie Robert et al., Degradation of an Fc-fusion recombinant protein by host cell proteases: Identification of a CHO cathepsin D protease, 104 BIOTECHNOLOGY AND BIOENGINEERING 1132-1141 (2009)). HCPs may present the greatest risk for persisting through purification operations into the final drug product. During long-term storage, the critical quality attributes of the product molecule must be maintained and degradation of excipients in the final product formulation must be minimized.

Several drug formulations on the market comprise polysorbate as one of the most commonly used nonionic surfactants in biopharmaceutical protein formulation that can improve protein stability and protect drug products from aggregation and denaturation (Sylvia Kiese et al., Shaken, Not Stirred: Mechanical Stress Testing of an IgG1 Antibody, 97 JOURNAL OF PHARMACEUTICAL SCIENCES 4347-4366 (2008); Ariadna Martos et al., Trends on Analytical Characterization of Polysorbates and Their Degradation Products in Biopharmaceutical Formulations, 106 JOURNAL OF PHARMACEUTICAL SCIENCES 1722-1735 (2017)). Polysorbate 20 (PS20) and polysorbate 80 (PS80) are the most commonly used nonionic surfactants in biopharmaceutical protein formulation that can improve protein stability and protect drug products from aggregation and denaturation. Typical polysorbate concentrations in drug products range can be between about 0.001% to about 0.1% (w/v) to provide sufficient efforts on protein stability.

Polysorbates, however, are liable to degradation that can drive undesired particulate formation in the formulated drug substances. Polysorbates are known to degrade in two main pathways: auto-oxidation and hydrolysis. Oxidation was found to be more likely to take place in PS80 due to the high content in unsaturated fatty acid ester substituents, whereas in PS20, oxidation was believed to take place on ether bond in polyoxyethylene chain which is not frequently observed (Oleg V. Borisov, Junyan A. Ji & Y. John Wang, Oxidative Degradation of Polysorbate Surfactants Studied by Liquid Chromatography-Mass Spectrometry, 104 JOURNAL OF PHARMACEUTICAL SCIENCES 1005-1018 (2015); Anthony Tomlinson et al., Polysorbate 20 Degradation in Biopharmaceutical Formulations: Quantification of Free Fatty Acids, Characterization of Particulates, and Insights into the Degradation Mechanism, 12 MOLECULAR PHARMACEUTICS 3805-3815 (2015); Jia Yao et al., A Quantitative Kinetic Study of Polysorbate Autoxidation: The Role of Unsaturated Fatty Acid Ester Substituents, 26 PHARMACEUTICAL RESEARCH 2303-2313 (2009)). In addition, polysorbates can also undergo hydrolysis by breaking the fatty acid ester bond. The particulates originating on degradation of polysorbates can form visible or even sub-visible which can raise the potential for immunogenicity in patients and may have varying effects on the drug product quality. One such possible impurity could be fatty acid particles that are formed during manufacture, shipment, storage, handling or administration of drug formulations comprising polysorbate. The fatty acid particles could potentially cause adverse immunogenic effects and impact shelf life. Additionally, the degradation of polysorbates can also cause reduction in the total amount of surfactant in the formulation affecting the product's stability during its manufacturing, storage, handling, and administration.

Putative phospholipase B-like 2 (PLBD2) was the first host cell protein that had been published to show evidence of enzymatic hydrolysis of PS20 (Nitin Dixit et al., Residual Host Cell Protein Promotes Polysorbate 20 Degradation in a Sulfatase Drug Product Leading to Free Fatty Acid Particles, 105 JOURNAL OF PHARMACEUTICAL SCIENCES 1657-1666 (2016)). The major evidence in the study was demonstrated by significant greater loss of PS20 when commercial recombinant human PLBD2 was spiked in. However, as the commercial PLBD2 only had a purity of ˜90%, one cannot rule out that other lipase impurities in the recombinant PLBD2 might be the root cause for PS20 degradation instead of PLBD2 itself. The present invention discloses steps and methods used to verify the role of PLBD2 in degrading polysorbates and identifying new lipases/esterase that could be responsible for polysorbate degradation. See Examples 13-18 which show that PLBD2 is not responsible for polysorbate degradation in monoclonal antibody drug products

Lipoprotein lipase (LPL) was also reported to be one of the host cell proteins that associated with PS20 and PS80 degradation and LPL knockout CHO cells demonstrated significant decrease on polysorbate degradation (Josephine Chiu et al., Knockout of a difficult-to-remove CHO host cell protein, lipoprotein lipase, for improved polysorbate stability in monoclonal antibody formulations, 114 BIOTECHNOLOGY AND BIOENGINEERING 1006-1015 (2016)). Group XV lysosomal phospholipase A₂ isomer X1 (LPLA₂) demonstrated the ability to degrade PS20 and PS80 at less than 1 ppm (Troii Hall et al., Polysorbates 20 and 80 Degradation by Group XV Lysosomal Phospholipase A 2 Isomer X1 in Monoclonal Antibody Formulations, 105 JOURNAL OF PHARMACEUTICAL SCIENCES 1633-1642 (2016)). Porcine liver esterase was reported to be able to specifically hydrolysis of polysorbate 80 (not PS20) and lead the formation of PS85 over time in mAb drug product (Steven R. Labrenz, Ester Hydrolysis of Polysorbate 80 in mAb Drug Product: Evidence in Support of the Hypothesized Risk After the Observation of Visible Particulate in mAb Formulations, 103 JOURNAL OF PHARMACEUTICAL SCIENCES 2268-2277 (2014)). Recently, a range of carboxyesters, including Pseudomonas cepacia lipase on immobead 150 (PCL), Candida antarctica lipase B on immobead 150 (CALB), Thermomyces lanuginosus lipase on immobead 150 (TLL), rabbit liver esterase (RLE), Candida antarctica lipase B (CALB) and porcine pancreatic lipase type II (PPL), were selected to study the hydrolysis of two unique PS20 and PS80 which contained 99% of laurate and 98% oleate esters, respectively. Different carboxyesters showed their unique degradation patterns, indicating that degradation pattern can be used to differentiate enzymes that hydrolyze polysorbates (A. C. Mcshan et al., Hydrolysis of Polysorbate 20 and 80 by a Range of Carboxylester Hydrolases, 70 PDA JOURNAL OF PHARMACEUTICAL SCIENCE AND TECHNOLOGY 332-345 (2016)). It can be essential to evaluate the effect of a host-cell protein co-purified with a drug product on polysorbates to ensure stability of the drug formulation. This can require identification of the host-cell protein and its ability to degrade polysorbates. Identification of host-cell proteins can be particularly challenging since the presence of HCPs is generally in ppm range which makes the isolation and identification of the HCP difficult.

The present invention discloses improved compositions comprising polysorbate with reduced level of host-cell proteins that can degrade polysorbate, methods for detection of such host-cell proteins and methods for preparing the compositions with reduced level such host-cell proteins.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

In some exemplary embodiments, the disclosure provides a composition comprising a protein of interest, surfactant, and a residual amount of a host-cell protein.

As used herein, the term “composition” refers to an active pharmaceutical agent that is formulated together with one or more pharmaceutically acceptable vehicles.

As used herein, the term “an active pharmaceutical agent” can include a biologically active component of a drug product. An active pharmaceutical agent can refer to any substance or combination of substances used in a drug product, intended to furnish pharmacological activity or to otherwise have direct effect in the diagnosis, cure, mitigation, treatment or prevention of disease, or to have direct effect in restoring, correcting or modifying physiological functions in animals. Non-limiting methods to prepare an active pharmaceutical agent can include using fermentation process, recombinant DNA, isolation and recovery from natural resources, chemical synthesis, or combinations thereof.

In some exemplary embodiments, the amount of active pharmaceutical agent in the formulation can range from about 0.01 mg/mL to about 600 mg/mL. In some specific embodiments, the amount of active pharmaceutical agent in the formulation can be about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 5 mg/mL, about 80 mg/mL, about 85 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 225 mg/mL, about 250 mg/mL, about 275 mg/mL, about 300 mg/mL, about 325 mg/mL, about 350 mg/mL, about 375 mg/mL, about 400 mg/mL, about 425 mg/mL, about 450 mg/mL, about 475 mg/mL, about 500 mg/mL, about 525 mg/mL, about 550 mg/mL, about 575 mg/mL, or about 600 mg/mL.

In some exemplary embodiments, pH of the composition can be greater than about 5.0. In one exemplary embodiment, the pH can be greater than about 5.0, greater than about 5.5, greater than about 6, greater than about 6.5, greater than about 7, greater than about 7.5, greater than about 8, or greater than about 8.5.

In some exemplary embodiments, the active pharmaceutical agent can be a protein of interest.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides' refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may contain one or multiple polypeptides to form a single functioning biomolecule. A protein can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation,” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012)). In some embodiments, proteins comprise modifications, adducts, and other covalently linked moieties. These modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as, nucleoproteins, glycoproteins, mucoproteins, chromoproteins, phosphoproteins, metalloproteins, and lipoproteins; and derived proteins, such as, primary derived proteins and secondary derived proteins.

In some exemplary embodiments, the protein of interest can be an antibody, a bispecific antibody, a multispecific antibody, antibody fragment, monoclonal antibody, fusion protein, and combinations thereof.

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment contains sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The phrase “bispecific antibody” includes an antibody capable of selectively binding two or more epitopes. Bispecific antibodies generally comprise two different heavy chains, with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain.

A typical bispecific antibody has two heavy chains each having three heavy chain CDRs, followed by a C_{H}1 domain, a hinge, a C_{H}2 domain, and a C_{H}3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats, such as, but not limited to triomab, knobs into holes IgG (kih IgG), crossMab, orth-Fab IgG, Dual-variable domains Ig (DVD-Ig), Two-in-one or dual action Fab (DAF), IgG-single-chain Fv (IgG-scFv), or κλ-bodies. The non-IgG-like different formats include Tandem scFvs, Diabody format, Single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014)).

The methods of producing BsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/22343 filed Jul. 29, 2016; and U.S. Ser. No. 15/814,095, filed Nov. 15, 2017. Low levels of homodimer impurities can be present at several steps during the manufacturing of bispecific antibodies. The detection of such homodimer impurities can be challenging when performed using intact mass analysis due to low abundances of the homodimer impurities and the co-elution of these impurities with main species when carried out using a regular liquid chromatographic method.

As used herein “multispecific antibody” or “Mab” refers to an antibody with binding specificities for at least two different antigens. While such molecules normally will only bind two antigens (i.e. bispecific antibodies, BsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, the protein of interest can have a pI in the range of about 4.5 to about 9.0. In one exemplary specific embodiment, the pI can be about 4.5, about 5.0, about 5.5, about 5.6, about 5.7, about 5.8, about 5.9, about 6.0, about 6.1 about 6.2, about 6.3, about 6.4, about 6.5, about 6.6, about 6.7, about 6.8, about 6.9, about 7.0, about 7.1 about 7.2, about 7.3, about 7.4, about 7.5, about 7.6, about 7.7, about 7.8, about 7.9, about 8.0, about 8.1 about 8.2, about 8.3, about 8.4, about 8.5, about 8.6, about 8.7, about 8.8, about 8.9, or about 9.0.

In some aspects, the types of protein of interest in the compositions can be at least two. In some aspects, one of the at least two protein of interest can be a monoclonal antibody, a polyclonal antibody, a bispecific antibody, an antibody fragment, a fusion protein, or an antibody-drug complex. In some other aspects, concentration of one of the at least two protein of interest can be about 20 mg/mL to about 400 mg/mL. In some exemplary aspects, the types of protein of interest in the compositions are two. In some exemplary aspects, the types of protein of interest in the compositions are three. In some exemplary aspects, the types of protein of interest in the compositions are five.

In other exemplary aspects, the two or more proteins of interest in the composition can be selected from trap proteins, chimeric receptor Fc-fusion proteins, chimeric proteins, antibodies, monoclonal antibodies, polyclonal antibodies, human antibodies, bispecific antibodies, multispecific antibodies, antibody fragments, nanobodies, recombinant antibody chimeras, cytokines, chemokines, or peptide hormones.

In some aspects, the composition can be a co-formulation.

In some exemplary embodiments, the protein of interest can be purified from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

In some exemplary aspects, the mammalian cells can be SIAE-knockout cells. In some other exemplary aspects, the mammalian cells can be LIPA-knockout cells. Targeted gene disruption or knockout can be achieved using zinc finger nucleases (ZFNs), transcription activator-like effector nucleases (TALENs), and clustered regularly interspaced short palindromic repeats (CRISPRs) technologies. Several groups have demonstrated the application of gene disruption technologies in CHO cells (Lise Marie Gray et al., One-step generation of triple knockout CHO cell lines using CRISPR/Cas9 and fluorescent enrichment, 10 BIOTECHNOLOGY JOURNAL 1446-1456 (2015); Carlotta Ronda et al., Accelerating genome editing in CHO cells using CRISPR Cas9 and CRISPy, a web-based target finding tool, 111 BIOTECHNOLOGY AND BIOENGINEERING 1604-1616 (2014); Tao Sun et al., Functional knockout of FUT8 in Chinese hamster ovary cells using CRISPR/Cas9 to produce a defucosylated antibody, 15 ENGINEERING IN LIFE SCIENCES 660-666 (2015)). Recent advances in the sequencing of the CHO-K1 and the Chinese hamster genome (Karina Brinkrolf et al., Chinese hamster genome sequenced from sorted chromosomes, 31 NATURE BIOTECHNOLOGY 694-695 (2013); Xun Xu et al., The genomic sequence of the Chinese hamster ovary (CHO)-K1 cell line, 29 NATURE BIOTECHNOLOGY 735-741 (2011)) have aided the rational design of engineered CHO cell lines with desired properties. A CHO-SIAE knockout or a CHO-LIPA knockout can be prepared on following the methods mentioned in the above-mentioned references or on following the procedure by Chiu et al. (Josephine Chiu et al., Knockout of a difficult-to-remove CHO host cell protein, lipoprotein lipase, for improved polysorbate stability in monoclonal antibody formulations, 114 BIOTECHNOLOGY AND BIOENGINEERING 1006-1015 (2016)).

In some specific exemplary aspects, the mammalian cells can be CHO-SIAE knockout cells. In some other specific exemplary aspects, the mammalian cells can be CHO-LIPA knockout cells.

In some exemplary aspects, the SIAE-knockout cells or LIPA-knockout cells can be obtained using ZFNs or transcription activator-like effector nucleases TALENs. These technologies use a common strategy of tethering endonuclease catalytic domains to modular DNA-binding proteins for inducing targeted DNA double stranded breaks (DSB) at specific genomic loci.

In some exemplary aspects, the SIAE-knockout cells or LIPA-knockout can be obtained using CRISPR technology. The knockout cells can be generated by co-expressing an endonuclease like Cas9 or Cas12a (also known as Cpf1) and a gRNA specific to the targeted gene.

The CRISPR can be an RNA-guided DNA endonuclease, catalyzes the double strand break (DSB) of DNA at the binding site of its RNA guide. The RNA guide can consist of a 42-nucleotide CRISPR RNA (crRNA) that joins with an 87-nucleotide trans-activating RNA (tracrRNA). The tracrRNA is complementary to and base pairs with the crRNA to form a functional crRNA/tracrRNA guide. This duplex RNA becomes bound to the Cas9 protein to form an active ribonucleoprotein (RNP) that can interrogate the genome for complementarity with the 20-nucleotide guide portion of the crRNA. A secondary requirement for strand breakage is that the Cas9 protein must recognize a protospacer adjacent motif (PAM) directly adjacent to the sequence complementary to the guide portion of crRNA (the crRNA target sequence). Alternatively, an active RNP complex can also be formed by replacing the crRNA/tracrRNA duplex with a single guide RNA (sgRNA) formed by covalently joining the crRNA and the tracrRNA. This sgRNA can be formed by fusing the twenty nucleotide guide portion of the crRNA directly to the processed tracrRNA sequence. The sgRNA can interact with both the Cas9 protein and the DNA in the same way and with similar efficiency as the crRNA/tracrRNA duplex would. The CRISPR bacterial natural defense mechanism has been shown to function effectively in mammalian cells and to activate break induced endogenous repair pathways. When a double strand break occurs in the genome, repair pathways will attempt to fix the DNA by either the canonical or alternative non-homologous end joining (NHEJ) pathways or homologous recombination, also referred to as homology-directed repair (HDR) if an appropriate template is available. A person skilled in the art can leverage these pathways to facilitate site specific deletion of genomic regions or insertion of exogenous DNA or HDR in mammalian cells.

In some exemplary aspects, the SIAE-knockout cells or LIPA knockout can be obtained using CRISPR/Cas9 technology. Like ZFNs and TALENs, Cas9 can promote genome editing by stimulating DSB at the target genomic loci. Upon cleavage by Cas9, the target locus undergoes one of two major pathways for DNA damage repair, the error-prone non-homologous end joining (NHEJ) or the high-fidelity homology directed repair (HDR) pathway. One or both pathways may be utilized to achieve the desired editing outcome. In some exemplary embodiments, the genomic target can be any nucleotide DNA sequence, such that the sequence is unique compared to the rest of the genome and the target is present immediately adjacent to a Protospacer Adjacent Motif (PAM). The guide RNA can contain a sequence complementary to the target DNA site, which directs the Cas to where it will cut. Cas9 from Streptococcus pyogenes (SpCas9) can be the endonuclease used for CRISPR editing. Once bound to the target, Cas9 “cuts” the DNA double helix, making a double-strand break (DSB). Some of the methods of using CRISPR/Cas9 technology are described in detail in the U.S. Pat. Publication No. 2016/0153005, which is incorporated by reference in its entirety. Some other methods of using CRISPR technology are described in detail in the U.S. Pat. Publication No. 2019/0032156, which is incorporated by reference in its entirety. Campenhout et al., which is also incorporated by reference in its entirety, provides guidelines to prepare a gene knockout using CRISPR/Cas9 (Claude Van Campenhout et al., Guidelines for optimized gene knockout using CRISPR/Cas9, 66 BIOTECHNIQUES 295-302 (2019)). Also with respect to general information on CRISPR-Cas Systems, details can be found in L. Cong et al., Multiplex Genome Engineering Using CRISPR/Cas Systems, 339 SCIENCE 819-823 (2013); Wenyan Jiang et al., RNA-guided editing of bacterial genomes using CRISPR-Cas systems, 31 NATURE BIOTECHNOLOGY 233-239 (2013); Haoyi Wang et al., One-Step Generation of Mice Carrying Mutations in Multiple Genes by CRISPR/Cas-Mediated Genome Engineering, 153 CELL 910-918 (2013); Silvana Konermann et al., Optical control of mammalian endogenous transcription and epigenetic states, 500 NATURE 472-476 (2013); F. Ann Ran et al., Double Nicking by RNA-Guided CRISPR Cas9 for Enhanced Genome Editing Specificity, 154 CELL 1380-1389 (2013); Patrick D Hsu et al., DNA targeting specificity of RNA-guided Cas9 nucleases, 31 NATURE BIOTECHNOLOGY 827-832 (2013); F Ann Ran et al., Genome engineering using the CRISPR-Cas9 system, 8 NATURE PROTOCOLS 2281-2308 (2013); Ophir Shalem et al., Genome-Scale CRISPR-Cas9 Knockout Screening in Human Cells, 343 SCIENCE 84-87 (2013); H. Nishimasu, R. Ishitani & O. Nureki, Crystal structure of Streptococcus pyogenes Cas9 in complex with guide RNA and target DNA, 156 CELL 935-949 (2014); Xuebing Wu et al., Genome-wide binding of the CRISPR endonuclease Cas9 in mammalian cells, 32 NATURE BIOTECHNOLOGY 670-676 (2014); and Patrick D. Hsu, Eric S. Lander & Feng Zhang, Development and Applications of CRISPR-Cas9 for Genome Engineering, 157 CELL 1262-1278 (2014), each of which is incorporated herein by reference.

In some exemplary aspects, the SIAE-knockout cells can be obtained by CRISPR/Cas9 technology on using sgRNA expression plasmids targeting either two or three sites in SIAE. Exemplary targeting guides can include A, B, and C, wherein A, B, and C can be 5′-ACTGCAGGTATGTGAGTGCT-3′ (SEQ ID NO.: 5) (nucleotides 538-545 of exon sequence, continues into intron, antisense strand), 5′-GGATTACGAATGTCACCCTG-3′ (SEQ ID NO.: 6) (nucleotides 314-333, sense strand), and 5′-TTGGGGAGGTAAGTGTACGT-3′ (SEQ ID NO.: 7) (nucleotides 784-794 of exon sequence, continues into intron, antisense strand).

In some exemplary aspects, the LIPA-knockout cells can be obtained by CRISPR/Cas9 technology on using sgRNA expression plasmids targeting at either two or three sites in LAL. Exemplary targeting guides can include 5′-GTACTGGGGATACCCGAGTG-3′ (SEQ ID NO.: 8) (nucleotides 120-139, sense strand) and 5′-CCAGTTGTCTATCTTCAGCA-3′ (SEQ ID NO.: 9) (nucleotides 232-251, sense strand).

In some embodiments, the composition can further comprise excipients including, but not limited to buffering agents, bulking agents, tonicity modifiers, solubilizing agents, and preservatives. Other additional excipients can also be selected based on function and compatibility with the formulations may be found, for example in REMINGTON: THE SCIENCE AND PRACTICE OF PHARMACY, (2005); U. S. Pharmacopeia: National formulary; LOUIS SANFORD GOODMAN ET AL., GOODMAN & GILMANS THE PHARMACOLOGICAL BASIS OF THERAPEUTICS (2001); KENNETH E. AVIS, HERBERT A. LIEBERMAN & LEON LACHMAN, PHARMACEUTICAL DOSAGE FORMS: PARENTERAL MEDICATIONS (1992); Praful Agrawala, Pharmaceutical Dosage Forms: Tablets. Volume 1, 79 Journal of Pharmaceutical Sciences 188 (1990); HERBERT A. LIEBERMAN, MARTIN M. RIEGER & GILBERT S. BANKER, PHARMACEUTICAL DOSAGE FORMS: DISPERSE SYSTEMS (1996); MYRA L. WEINER & LOIS A. KOTKOSKIE, EXCIPIENT TOXICITY AND SAFETY (2000), herein incorporated by reference in their entirety.

In some exemplary aspects, the composition can be stable. The stability of a composition can comprise evaluating the chemical stability, physical stability or functional stability of the active pharmaceutical agent. The formulations of the present invention typically exhibit high levels of stability of the active pharmaceutical agent.

In terms of protein formulations, the term “stable,” as used herein refers to the protein of interest within the formulations being able to retain an acceptable degree of chemical structure or biological function after storage under exemplary conditions defined herein. A formulation may be stable even though the protein of interest contained therein does not maintain 100% of its chemical structure or biological function after storage for a defined amount of time. Under certain circumstances, maintenance of about 90%, about 95%, about 96%, about 97%, about 98% or about 99% of a protein's structure or function after storage for a defined amount of time may be regarded as “stable”.

Stability can be measured, inter alia, by determining the percentage of native protein(s) that remain in the formulation after storage for a defined amount of time at a defined temperature. The percentage of native protein can be determined by, inter alia, size exclusion chromatography (e.g., size exclusion high performance liquid chromatography [SE-HPLC]), such that native means non-aggregated and non-degraded. An “acceptable degree of stability,” as that phrase is used herein, means that at least 90% of the native form of the protein can be detected in the formulation after storage for a defined amount of time at a given temperature. In certain embodiments, at least about 90%, 91%, 92%, 93%, 94%, 95%, 96%, 97%, 98%, 99% or 100% of the native form of the protein can be detected in the formulation after storage for a defined amount of time at a defined temperature. The defined amount of time after which stability is measured can be at least 14 days, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more.

Stability can be measured, inter alia, by determining the percentage of protein that forms in an aggregate within the formulation after storage for a defined amount of time at a defined temperature, wherein stability is inversely proportional to the percent aggregate that is formed. This form of stability is also referred to as “colloidal stability” herein. The percentage of aggregated protein can be determined by, inter alia, size exclusion chromatography (e.g., size exclusion high performance liquid chromatography [SE-HPLC]). An “acceptable degree of stability,” as that phrase is used herein, means that at most 6% of the protein is in an aggregated form detected in the formulation after storage for a defined amount of time at a given temperature. In certain embodiments an acceptable degree of stability means that at most about 6%, 5%, 4%, 3%, 2%, 1%, 0.5%, or 0.1% of the protein can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 1 1 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4° C., about 5° C., about 25° C., about 35° C., about 37° C. or about 45° C. For example, a pharmaceutical formulation may be deemed stable if after six months of storage at 5° C., less than about 3%, 2%, 1%, 0.5%, or 0.1% of the protein is detected in an aggregated form. A pharmaceutical formulation may also be deemed stable if after six months of storage at 25° C., less than about 4%, 3%, 2%, 1%, 0.5%, or 0.1% of the protein is detected in an aggregated form. A pharmaceutical formulation may also be deemed stable if after 28 days of storage at 45° C., less than about 6%, 5%, 4%, 3%, 2%, 1%, 0.5%, or 0.1% of the protein is detected in an aggregated form. A pharmaceutical formulation may also be deemed stable if after three months of storage at −20° C., −30° C., or −80° C. less than about 3%, 2%, 1%, 0.5%, or 0.1% of the protein is detected in an aggregated form.

Stability can also be measured, inter alia, by determining the percentage of protein that forms in an aggregate within the formulation after storage for a defined amount of time at a defined temperature, wherein stability is inversely proportional to the percent aggregate that is formed. This form of stability is also referred to as “colloidal stability” herein. The percentage of aggregated protein can be determined by, inter alia, size exclusion chromatography (e.g., size exclusion high performance liquid chromatography [SE-HPLC]). An acceptable degree of stability,” as that phrase is used herein, means that at most 6% of the protein is in an aggregated form detected in the formulation after storage for a defined amount of time at a given temperature. In certain embodiments an acceptable degree of stability means that at most about 6%, 5%, 4%, 3%, 2%, 1%, 0.5%, or 0.1% of the protein can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 1 1 months, at least 12 months, at least 18 months, at least 24 months, or more. The temperature at which the pharmaceutical formulation may be stored when assessing stability can be any temperature from about −80° C. to about 45° C., for example, storage at about −80° C., about −30° C., about −20° C., about 0° C., about 4°-8° C., about 5° C., about 25° C., about 35° C., about 37° C. or about 45° C. For example, a pharmaceutical formulation may be deemed stable if after six months of storage at 5° C., less than about 3%, 2%, 1%, 0.5%, or 0.1% of the protein is detected in an aggregated form. A pharmaceutical formulation may also be deemed stable if after six months of storage at 25° C., less than about 4%, 3%, 2%, 1%, 0.5%, or 0.1% of the protein is detected in an aggregated form. A pharmaceutical formulation may also be deemed stable if after 28 days of storage at 45° C., less than about 6%, 5%, 4%, 3%, 2%, 1%, 0.5%, or 0.1% of the protein is detected in an aggregated form. A pharmaceutical formulation may also be deemed stable if after three months of storage at −20° C., −30° C., or −80° C. less than about 3%, 2%, 1%, 0.5%, or 0.1% of the protein is detected in an aggregated form.

Stability can be also measured, inter alia, by determining the percentage of protein that migrates in a more acidic fraction during ion exchange (“acidic form”) than in the main fraction of protein (“main charge form”), wherein stability is inversely proportional to the fraction of protein in the acidic form. While not wishing to be bound by theory, deamidation of the protein may cause the protein to become more negatively charged and thus more acidic relative to the non-deamidated protein (see, e.g., Robinson, N. (2002) “Protein Deamidation” PNAS, 99(8):5283-5288). The percentage of “acidified” protein can be determined by, inter alia, ion exchange chromatography (e.g., cation exchange high performance liquid chromatography [CEX-HPLC]). An “acceptable degree of stability,” as that phrase is used herein, means that at most 49% of the protein is in a more acidic form detected in the formulation after storage for a defined amount of time at a defined temperature. In certain exemplary embodiments, an acceptable degree of stability means that at most about 49%, 45%, 40%, 35%, 30%, 25%, 20%, 15%, 10%, 5%, 4%, 3%, 2%, 1%, 0.5%, or 0.1% of the protein can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more.

The temperature at which the pharmaceutical formulation may be stored when assessing stability can be any temperature from about −80° C. to about 45° C., for example, storage at about −80° C., about −30° C., about −20° C., about 0° C., about 4°-8° C., about 5° C., about 25° C., or about 45° C. For example, a pharmaceutical formulation may be deemed stable if after three months of storage at −80° C., −30° C., or −20° C. less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the protein is in a more acidic form. A pharmaceutical formulation may also be deemed stable if after six months of storage at 5° C., less than about 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the protein is in a more acidic form. A pharmaceutical formulation may also be deemed stable if after six months of storage at 25° C., less than about 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the protein is in a more acidic form. A pharmaceutical formulation may also be deemed stable if after 28 days of storage at 45° C., less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the protein can be detected in a more acidic form.

Other methods may be used to assess the stability of the formulations of the present invention such as, for example, differential scanning calorimetry (DSC) to determine thermal stability, controlled agitation to determine mechanical stability, and absorbance at about 350 nm or about 405 nm to determine solution turbidities. For example, a formulation of the present invention may be considered stable if, after 6 or more months of storage at about 5° C. to about 25° C., the change in OD405 of the formulation is less than about 0.05 (e.g., 0.04, 0.03, 0.02, 0.01, or less) from the OD405 of the formulation at time zero. Measuring the biological activity or binding affinity of the protein to its target may also be used to assess stability. For example, a formulation of the present invention may be regarded as stable if, after storage at e.g., 5° C., 25° C., 45° C., etc. for a defined amount of time (e.g., 1 to 12 months), the protein contained within the formulation binds to its target with an affinity that is at least 90%, 95%, or more of the binding affinity of the protein prior to said storage. Binding affinity may be determined by e.g., ELISA or plasmon resonance. Biological activity may be determined by a protein activity assay, such as e.g., contacting a cell that expresses the protein with the formulation comprising the protein. The binding of the protein to such a cell may be measured directly, such as e.g., via FACS analysis. Alternatively, the downstream activity of the protein system may be measured in the presence of the protein and compared to the activity of the protein system in the absence of protein.

In some exemplary embodiments, the composition can be used for the treatment, prevention and/or amelioration of a disease or disorder. Exemplary, non-limiting diseases and disorders that can be treated and/or prevented by the administration of the pharmaceutical formulations of the present invention include, infections; respiratory diseases; pain resulting from any condition associated with neurogenic, neuropathic or nociceptic pain; genetic disorder; congenital disorder; cancer; herpetiformis; chronic idiopathic urticarial; scleroderma, hypertrophic scarring; Whipple's Disease; benign prostate hyperplasia; lung disorders, such as mild, moderate or severe asthma, allergic reactions; Kawasaki disease, sickle cell disease; Churg-Strauss syndrome; Grave's disease; pre-eclampsia; Sjogren's syndrome; autoimmune lymphoproliferative syndrome; autoimmune hemolytic anemia; Barrett's esophagus; autoimmune uveitis; tuberculosis; nephrosis; arthritis, including chronic rheumatoid arthritis; inflammatory bowel diseases, including Crohn's disease and ulcerative colitis; systemic lupus erythematosus; inflammatory diseases; HIV infection; AIDS; LDL apheresis; disorders due to PCSK9-activating mutations (gain of function mutations, “GOF”), disorders due to heterozygous Familial Hypercholesterolemia (heFH); primary hypercholesterolemia; dyslipidemia; cholestatic liver diseases; nephrotic syndrome; hypothyroidism; obesity; atherosclerosis; cardiovascular diseases; neurodegenerative diseases; neonatal Onset Multisystem Inflammatory Disorder (NOM ID/CINCA); Muckle-Wells Syndrome (MWS); Familial Cold Autoinflammatory Syndrome (FCAS); familial Mediterranean fever (FMF); tumor necrosis factor receptor-associated periodic fever syndrome (TRAPS); systemic onset juvenile idiopathic arthritis (Still's Disease); diabetes mellitus type 1 and type 2; auto-immune diseases; motor neuron disease; eye diseases; sexually transmitted diseases; tuberculosis; disease or condition which is ameliorated, inhibited, or reduced by a VEGF antagonist; disease or condition which is ameliorated, inhibited, or reduced by a PD-1 inhibitor; disease or condition which is ameliorated, inhibited, or reduced by a Interleukin antibody; disease or condition which is ameliorated, inhibited, or reduced by a NGF antibody; disease or condition which is ameliorated, inhibited, or reduced by a PCSK9 antibody; disease or condition which is ameliorated, inhibited, or reduced by a ANGPTL antibody; disease or condition which is ameliorated, inhibited, or reduced by an activin antibody; disease or condition which is ameliorated, inhibited, or reduced by a GDF antibody; disease or condition which is ameliorated, inhibited, or reduced by a Fel d 1 antibody; disease or condition which is ameliorated, inhibited, or reduced by a CD antibody; disease or condition which is ameliorated, inhibited, or reduced by a C5 antibody or combinations thereof.

In some exemplary embodiments, the composition can be administered to a patient. Administration may be via any route acceptable to those skilled in the art. Non-limiting routes of administration include oral, topical, or parenteral. Administration via certain parenteral routes may involve introducing the formulations of the present invention into the body of a patient through a needle or a catheter, propelled by a sterile syringe or some other mechanical device such as a continuous infusion system. A composition provided by the present invention may be administered using a syringe, injector, pump, or any other device recognized in the art for parenteral administration. A composition of the present invention may also be administered as an aerosol for absorption in the lung or nasal cavity. The compositions may also be administered for absorption through the mucus membranes, such as in buccal administration.

In some exemplary embodiments, the surfactant in the composition can be polysorbate. As used herein, “polysorbate” refers to a common excipient used in formulation development to protect antibodies against various physical stresses such as agitation, freeze-thaw processes, and air/water interfaces (Emily Ha, Wei Wang & Y. John Wang, Peroxide formation in polysorbate 80 and protein stability, 91 JOURNAL OF PHARMACEUTICAL SCIENCES 2252-2264 (2002); Bruce A. Kerwin, Polysorbates 20 and 80 Used in the Formulation of Protein Biotherapeutics: Structure and Degradation Pathways, 97 JOURNAL OF PHARMACEUTICAL SCIENCES 2924-2935 (2008); Hanns-Christian Mahler et al., Adsorption Behavior of a Surfactant and a Monoclonal Antibody to Sterilizing-Grade Filters, 99 Journal of Pharmaceutical Sciences 2620-2627 (2010)) and can include a non-ionic, amphipathic surfactant composed of fatty acid esters of polyoxyethylene-sorbitan. The esters can include polyoxyethylene sorbitan head group and either a saturated monolaurate side chain (polysorbate 20; PS20) or an unsaturated monooleate side chain (polysorbate 80; PS80). In some aspects, the polysorbate can be present in the formulation in the range of about 0.001% to 2% (weight/volume). Polysorbate can also contain a mixture of various fatty acid chains; for example, polysorbate 80 contains oleic, palmitic, myristic and stearic fatty acids, with the monooleate fraction making up approximately 58% of the polydisperse mixture (Nitin Dixit et al., Residual Host Cell Protein Promotes Polysorbate 20 Degradation in a Sulfatase Drug Product Leading to Free Fatty Acid Particles, 105 JOURNAL OF PHARMACEUTICAL SCIENCES 1657-1666 (2016)). Non-limiting examples of polysorbates include polysorbate-20, polysorbate-40, polysorbate-60, polysorbate-65, and polysorbate-80.

A polysorbate can be susceptible to auto-oxidation in a pH- and temperature-dependent manner, and additionally, exposure to UV light can also produce instability (Ravuri S.k. Kishore et al., Degradation of Polysorbates 20 and 80: Studies on Thermal Autoxidation and Hydrolysis, 100 JOURNAL OF PHARMACEUTICAL SCIENCES 721-731 (2011)), resulting in free fatty acids in solution along with the sorbitan head group. The free fatty acids resulting from polysorbate can include any aliphatic fatty acids with six to twenty carbons. Non-limiting examples of free fatty acids include oleic acid, palmitic acid, stearic acid, myristic acid, lauric acid, or combinations thereof.

In some exemplary aspects, the polysorbate can form free fatty acid particles. The free fatty acid particles can be at least 5 μm in size. Further, these fatty acid particles can be classified according to their size as visible (>100 μm), sub-visible (<100 μm, which can be sub-divided into micron (1-100 μm) and submicron (100 nm-1000 nm)) and nanometer particles (<100 nm) (Linda Narhi, Jeremy Schmit & Deepak Sharma, Classification of protein aggregates, 101 JOURNAL OF PHARMACEUTICAL SCIENCES 493-498). In some exemplary aspects, the fatty acid particles can be visible particles. Visible particles can be determined by visual inspection. In some exemplary embodiments, the fatty acid particles can be sub-visible particles. Subvisible particles can be monitored by the light blockage method according to United States Pharmacopeia (USP).

In some exemplary aspects, the concentration of polysorbate in the composition can be about 0.001% w/v, about 0.002% w/v, about 0.003% w/v, about 0.004% w/v, about 0.005% w/v, about 0.006% w/v, about 0.007% w/v, about 0.008% w/v, about 0.009% w/v, about 0.01% w/v, about 0.011% w/v, about 0.015% w/v, about 0.02% w/v, 0.025% w/v, about 0.03% w/v, about 0.035% w/v, about 0.04% w/v, about 0.045% w/v, about 0.05% w/v, about 0.055% w/v, about 0.06% w/v, about 0.065% w/v, about 0.07% w/v, about 0.075% w/v, about 0.08% w/v, about 0.085% w/v, about 0.09% w/v, about 0.095% w/v, about 0.1% w/v, about 0.11% w/v, about 0.115% w/v, about 0.12% w/v, about 0.125% w/v, about 0.13% w/v, about 0.135% w/v, about 0.14% w/v, about 0.145% w/v, about 0.15% w/v, about 0.155% w/v, about 0.16% w/v, about 0.165% w/v, about 0.17% w/v, about 0.175% w/v, about 0.18% w/v, about 0.185% w/v, about 0.19% w/v, about 0.195% w/v, or about 0.2% w/v.

In some exemplary aspects, the polysorbate can be degraded by the host-cell protein present in the composition. As used herein, the term “host-cell protein” includes protein derived from the host cell and can be unrelated to the desired protein of interest. Host-cell protein can be a process-related impurity which can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

In some exemplary aspects, the host-cell protein can have a pI in the range of about 4.5 to about 9.0. In one exemplary specific embodiment, the pI can be about 4.5, about 5.0, about 5.5, about 5.6, about 5.7, about 5.8, about 5.9, about 6.0, about 6.1 about 6.2, about 6.3, about 6.4, about 6.5, about 6.6, about 6.7, about 6.8, about 6.9, about 7.0, about 7.1 about 7.2, about 7.3, about 7.4, about 7.5, about 7.6, about 7.7, about 7.8, about 7.9, about 8.0, about 8.1 about 8.2, about 8.3, about 8.4, about 8.5, about 8.6, about 8.7, about 8.8, about 8.9, or about 9.0.

In some exemplary aspects, the types of host-cell proteins in the composition can be at least two.

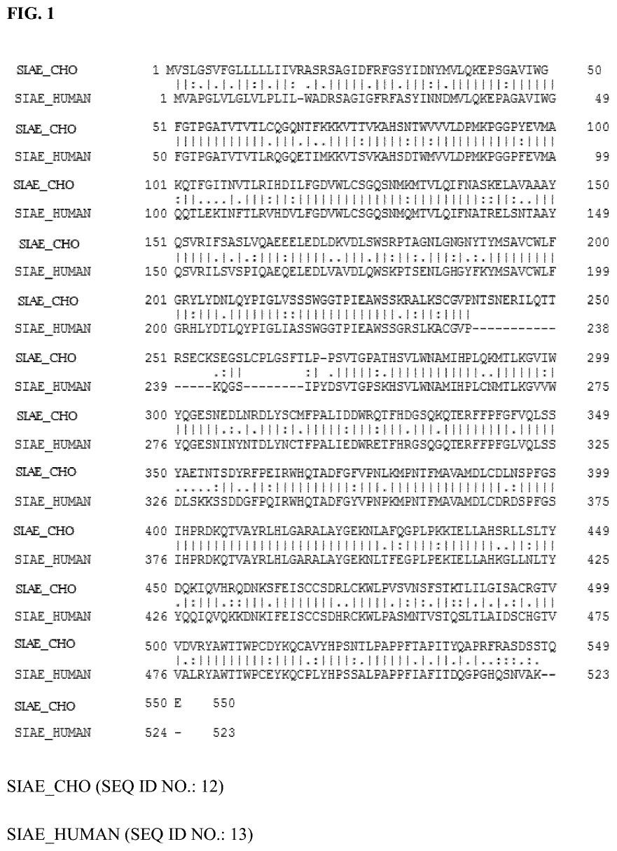

In some exemplary embodiments, the host-cell protein can be sialate o-acetylesterase. As used herein, “sialate o-acetylesterase” or “SIAE” are used interchangeably and refer to the enzyme which is encoded by the SIAE gene. Certain scientific publications on SIAE include Roland Schauer, Gerd Reuter & Sabine Stoll, Sialate O-acetylesterases: key enzymes in sialic acid catabolism, 70 BIOCHIMIE 1511-1519 (1988); Flavia Orizio et al., Human sialic acid acetyl esterase: Towards a better understanding of a puzzling enzyme, 25 GLYCOBIOLOGY 992-1006 (2015) and G. Vinayaga Srinivasan & Roland Schauer, Assays of sialate-O-acetyltransferases and sialate-O-acetylesterases, 26 GLYCOCONJUGATE JOURNAL 935-944 (2008). Sialate O-acetylesterase (SIAE) is an enzyme belongs to SGNH-hydrolase family with >7000 members and play important roles in a variety of biological events (Orizio et al, supra). The two isoforms of SIAE are the cytosolic sialic acid esterase (Cse) and the lysosomal sialic acid esterase (Lse). Lse is the isoform that is detected from most tissues. The most well-known function of SIAE is its esterase activity that acting on the hydroxyl groups in position 9 and 4 of sialic acid to remove acetyl moieties, however, several aspects of SIAE biology remain unclear (Srinivasan and Schauer, supra). In silico characterization and 3D structural modeling of SIAE protein demonstrated that SIAE is highly glycosylated and the glycosylation influence the biological activity of the enzyme (Orizio et al, supra). The amino acid sequence of the human RAE shows 69.13% homology to the amino acid sequence of the CHO SIAE (86.5% similarity) (See FIG. 1).

There exists a structural similarity between sialic acid and polysorbate POE head group suggests the possibility that SIAE can degrade polysorbates. Each polysorbate component can be hydrolyzed with different efficiency.

In some other exemplary embodiments, the host-cell protein can be lysosomal acid lipase. As used herein, “lysosomal acid lipase” or “LAL” are used interchangeably and refer to the enzyme which is a 378-amino acid protein that is expressed by all cell types and encoded by the LIPA gene on chromosome 10. As an enzyme, LAL breaks down fats (lipids) such as triglycerides and cholesteryl esters. LAL can also be referred to as cholesterol ester hydrolase, lipase A, or sterol esterase. The role of LAL in cellular lipid metabolism is detailed in M. Gomaraschi, F. Bonacina & G. d. Norata, Lysosomal Acid Lipase: From Cellular Lipid Handler to Immunometabolic Target, 40 TRENDS IN PHARMACOLOGICAL SCIENCES 104-115 (2019).

The effect of LAL on degradation of polysorbate was identified by using detecting methods according to some exemplary embodiments.

Having identified LAL and/or SIAE as HCPs that can degrade polysorbates in certain protein preparations, it would be highly advantageous and desirable to have reagents, methods, and kits for the specific, sensitive, and quantitative determination and/or depletion of LAL and/or SIAE levels, as well as to develop methods of preparing compositions with low levels of LAL and/or SIAE and/or by using LIPA and/or SIAE-knockout cell-line.

In some exemplary embodiments, the disclosure provides compositions which comprises less than about 5 ppm of a host-cell protein, wherein the host-cell protein can be SIAE or LAL.

In some exemplary aspects, the residual amount of SIAE in the composition can be less than about 5 ppm. In some specific exemplary aspects, the residual amount of SIAE is less than about 0.01 ppm, about 0.02 ppm, about 0.03 ppm, about 0.04 ppm, about 0.05 ppm, about 0.06 ppm, 0.07 ppm, 0.08 ppm, 0.09 ppm, about 0.1 ppm, about 0.2 ppm, about 0.3 ppm, about 0.4 ppm, about 0.5 ppm, about 0.6 ppm, 0.7 ppm, 0.8 ppm, 0.9 ppm, about 1 ppm, about 2 ppm, about 3 ppm, about 4 ppm, or about 5 ppm.

In some exemplary aspects, the residual amount of LAL in the composition can be less than about 5 ppm. In some specific exemplary aspects, the residual amount of LAL is less than about 0.01 ppm, about 0.02 ppm, about 0.03 ppm, about 0.04 ppm, about 0.05 ppm, about 0.06 ppm, 0.07 ppm, 0.08 ppm, 0.09 ppm, about 0.1 ppm, about 0.2 ppm, about 0.3 ppm, about 0.4 ppm, about 0.5 ppm, about 0.6 ppm, 0.7 ppm, 0.8 ppm, 0.9 ppm, about 1 ppm, about 2 ppm, about 3 ppm, about 4 ppm, or about 5 ppm.

In some exemplary aspects, the disclosure provides various methods of preparing a composition having a protein of interest which comprises less than about 5 ppm of a host-cell protein, wherein the host-cell protein can be SIAE or LAL.

In some exemplary aspects, a method of preparing the composition having a protein of interest with less than about 5 ppm of a host-cell protein can include forming a sample matrix with the protein of interest cultured using mammalian cells, contacting the sample matrix to a first chromatography resin and washing the bound protein of interest to form an eluate. In some specific exemplary aspects, the host-cell protein can be SIAE or LAL.

In another exemplary embodiment, the sample matrix can be obtained from any step of the bioprocess, such as, culture cell culture fluid (CCF), harvested cell culture fluid (HCCF), process performance qualification (PPQ), any step in the downstream processing, drug solution (DS), or a drug product (DP) comprising the final formulated product. In some other specific exemplary aspects, the sample matrix can be selected from any step of the downstream process of clarification, chromatographic purification, viral inactivation, or filtration. In some specific exemplary embodiments, the drug product can be selected from manufactured drug product in the clinic, shipping, storage, or handling.

In some exemplary aspects, the method of preparing the composition having a protein of interest with less than about 5 ppm of a host-cell protein can further include contacting the eluate to a second chromatography resin. In some specific exemplary embodiments, a flow-through from washing the second chromatography resin can be collected.

In some exemplary aspects, the method of preparing the composition having a protein of interest with less than about 5 ppm of a host-cell protein can further include contacting the flow-through to a third chromatography resin. In some specific exemplary embodiments, a second flow-through from washing the third chromatography resin can be collected.

The first chromatography resin, second chromatography resin and the third chromatography resin can be of same or different types. Non-limiting examples of the resins include affinity chromatography, ion-exchange chromatography, hydrophobic interaction chromatography, or mixed-mode chromatography.

In some exemplary embodiments, the chromatography method can be a liquid chromatography method. As used herein, the term “liquid chromatography” refers to a process in which a chemical mixture carried by a liquid can be separated into components as a result of differential distribution of the chemical entities as they flow around or over a stationary liquid or solid phase. Non-limiting examples of liquid chromatography include reverse phase liquid chromatography, ion-exchange chromatography, size exclusion chromatography, affinity chromatography, mixed-mode chromatography, hydrophobic chromatography or mixed-mode chromatography.

As used herein, “affinity chromatography” can include separations including any method by which two substances are separated based their affinity to chromatographic material. It can comprise subjecting the substances to a column comprising a suitable affinity chromatographic media. Non-limiting examples of such chromatographic media include, but are not limited to Protein A resin, Protein G resin, affinity supports comprising the antigen against which the binding molecule was raised, and affinity supports comprising an Fc binding protein. In one aspect, an affinity column can be equilibrated with a suitable buffer prior to sample loading. An example of a suitable buffer can be a Tris/NaCl buffer, pH around 7.2. Following this equilibration, the sample can be loaded onto the column. Following the loading of the column, the column can be washed one or multiple times using, e.g., the equilibrating buffer. Other washes including washes employing different buffers can be used before eluting the column. The affinity column can then be eluted using an appropriate elution buffer. An example of a suitable elution buffer can be an acetic acid/NaCl buffer, pH around 3.5. The eluate can be monitored using techniques well known to those skilled in the art. For example, the absorbance at OD280 can be followed.

As used herein, “ion exchange chromatography” can include separations including any method by which two substances are separated based on the difference in their respective ionic charges, either on the molecule of interest and/or chromatographic material as a whole or locally on specific regions of the molecule of interest and/or chromatographic material, and thus can employ either cationic exchange material or anionic exchange material. Ion exchange chromatography separates molecules based on differences between the local charges of the molecules of interest and the local charges of the chromatographic material. A packed ion-exchange chromatography column or an ion-exchange membrane device can be operated in a bind-elute mode, a flow-through, or a hybrid mode. After washing the column or the membrane device with the equilibration buffer or another buffer with different pH and/or conductivity, the product recovery can be achieved by increasing the ionic strength (i.e., conductivity) of the elution buffer to compete with the solute for the charged sites of the ion exchange matrix. Changing the pH and thereby altering the charge of the solute can be another way to achieve elution of the solute. The change in conductivity or pH may be gradual (gradient elution) or stepwise (step elution). The column can be then regenerated before next use. Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substituents include carboxymethyl (CM), sulfoethyl (SE), sulfopropyl (SP), phosphate (P) and sulfonate (S). Cellulose ion exchange medias or support can include DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow, and Capto™ S are all available from GE Healthcare. Further, both DEAE and CM derivitized ethylene glycol-methacrylate copolymer such as TOYOPEARL™ DEAE-650S or M and TOYOPEARL™ CM-650S or M are available from Toso Haas Co., Philadelphia, Pa., or Nuvia S and UNOSphere™ S from BioRad, Hercules, Calif, Eshmuno® S from EMD Millipore, Mass.

As used herein, the term “hydrophobic interaction chromatography resin” can include a solid phase which can be covalently modified with phenyl, octyl, or butyl chemicals. It can use the properties of hydrophobicity to separate molecules from one another. In this type of chromatography, hydrophobic groups such as, phenyl, octyl, hexyl or butyl can be attached to the stationary column. Molecules that pass through the column that have hydrophobic amino acid side chains on their surfaces are able to interact with and bind to the hydrophobic groups on the column. Examples of hydrophobic interaction chromatography resins or support include Phenyl sepharose FF, Capto Phenyl (GE Healthcare, Uppsala, Sweden), Phenyl 650-M (Tosoh Bioscience, Tokyo, Japan) and Sartobind Phenyl (Sartorius corporation, New York, USA).

As used herein, the term “Mixed Mode Chromatography (MMC)” or “multimodal chromatography” includes a chromatographic method in which solutes interact with stationary phase through more than one interaction mode or mechanism. MMC can be used as an alternative or complementary tool to traditional reversed-phased (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Mixed mode chromatography media can provide unique selectivity that cannot be reproduced by single mode chromatography. Mixed mode chromatography can also provide potential cost savings, longer column lifetimes and operation flexibility compared to affinity-based methods. In some exemplary embodiments, the mixed mode chromatography media can be comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In some specific exemplary aspects, the support can be prepared from a native polymer, such as cross-linked carbohydrate material, such as agarose, agar, cellulose, dextran, chitosan, konjac, carrageenan, gellan, alginate and the like. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964). Alternatively, the support can be prepared from a synthetic polymer, such as cross-linked synthetic polymers, for example, styrene or styrene derivatives, divinylbenzene, acrylamides, acrylate esters, methacrylate esters, vinyl esters, vinyl amides and the like. Such synthetic polymers can be produced according to standard methods, see e.g., “Styrene based polymer supports developed by suspension polymerization” (R Arshady: Chimica e L'Industria 70(9), 70-75 (1988)). Porous native or synthetic polymer supports are also available from commercial sources, such as Amersham Biosciences, Uppsala, Sweden.

In some exemplary embodiments, the method of preparing the composition having a protein of interest with less than about 5 ppm of a host-cell protein can further include filtering one or all of the following: sample matrix, eluate, flow-through, or second flow-through by viral filtration.

As used herein, “viral filtration” can include filtration using suitable filters including, but not limited to, Planova 20N™, 50 N or BioEx from Asahi Kasei Pharma, Viresolve™ filters from EMD Millipore, ViroSart CPV from Sartorius, or Ultipor DV20 or DV50™ filter from Pall Corporation. It will be apparent to one of ordinary skill in the art to select a suitable filter to obtain desired filtration performance.

In some exemplary aspects, the method of preparing the composition having a protein of interest with less than about 5 ppm of a host-cell protein can further include filtering one or all of the following: sample matrix, eluate, flow-through, second flow-through, filtrate on viral filtration to UF/DF procedure.

As used herein, the term “ultrafiltration” or “UF” can include a membrane filtration process similar to reverse osmosis, using hydrostatic pressure to force water through a semi-permeable membrane. Ultrafiltration is described in detail in: LEOS J. ZEMAN & ANDREW L. ZYDNEY, MICROFILTRATION AND ULTRAFILTRATION: PRINCIPLES AND APPLICATIONS (1996). Filters with a pore size of smaller than 0.1 μm can be used for ultrafiltration. By employing filters having such small pore size, the volume of the sample can be reduced through permeation of the sample buffer through the filter while antibodies are retained behind the filter.

As used herein, “diafiltration” or “DF” can include a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultrafiltered at a rate approximately equal to the ultrafiltration rate. This washes microspecies from the solution at a constant volume, effectively manufacturing the retained antibody. In certain embodiments of the present invention, a diafiltration step can be employed to exchange the various buffers used in connection with the instant invention, optionally prior to further chromatography or other purification steps, as well as to remove impurities from the antibody preparation.

In some exemplary aspects, the method of preparing the composition having a protein of interest with less than about 5 ppm of a host-cell protein can further include contacting one of the following: sample matrix, eluate, flow-through, second flow-through, filtrate on viral filtration, or filtrate on UF/DF procedure to a bead having anti-HCP antibody. In some aspects, the ratio of amount of anti-HCP antibody to amount of the bead can range from about 1 μg/g to about 50 μg/g. For example, in some aspects, the ratio can be about 1 μg/g, about 2 μg/g, about 3 μg/g, about 4 μg/g, about 5 μg/g, about 6 μg/g, about7 μg/g, about 8 μg/g, about 9 μg/g, about 10 μg/g, about 15 μg/g, about 20 μg/g, about 25 μg/g, about 30 μg/g, about 35 μg/g, about 40 μg/g, about 45 μg/g, or about 50 μg/g. In some aspects, the beads can include polymer particles with a defined surface for adsorption of a biological molecule. In some specific embodiments, the beads can have a superparamagnetic property.

In some exemplary aspects, the anti-HCP antibody can be anti-SIAE antibody. In some other exemplary aspects, the anti-HCP antibody can be anti-LAL antibody. The anti-SAIE antibody or the anti-LAL antibody can be of the same origin as the cells used to manufacture the protein of interest of the composition are. In some exemplary aspects, the anti-HCP antibody can be of human origin. In some exemplary aspects, the anti-HCP antibody can be of hamster origin. In some exemplary aspects, the method can further include washing the beads with a wash buffer. In some exemplary aspects, the method can optionally further include collecting wash fractions from the washing step. Example of one such kit that can be used to produce anti-SAIE antibody or anti-LAL antibody can the Dynabeads™ Antibody Coupling Kit.

In some exemplary embodiments, the disclosure provides various methods of detecting HCP in a sample matrix, comprising contacting the sample matrix with a biotinylated anti-HCP antibody and incubating the sample matrix with a resin, performing elution on the resin to form an eluate, adding hydrolyzing agent to the eluate to obtain digests and analyzing the digests to detect the HCP. In some specific exemplary embodiments, the HCP can be SIAE or LAL.

In some exemplary aspects, the resin can include a bead with an ability to adsorb the biotinylated anti-HCP antibody. In some specific exemplary embodiments, the bead can be a magnetic bead.

In some specific exemplary aspects, the elution can be performed using one or more solvents selected from acetonitrile, water and acetic acid.

As used herein, the term “hydrolyzing agent” refers to any one or combination of a large number of different agents that can perform digestion of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. Non-limiting examples of hydrolyzing agents that can carry out non-enzymatic digestion include the use of high temperature, microwave, ultrasound, high pressure, infrared, solvents (non-limiting examples are ethanol and acetonitrile), immobilized enzyme digestion (IMER), magnetic particle immobilized enzymes, and on-chip immobilized enzymes. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)). One or a combination of hydrolyzing agents can cleave peptide bonds in a protein or polypeptide, in a sequence-specific manner, generating a predictable collection of shorter peptides.

The ratio of hydrolyzing agent to the protein and the time required for digestion can be appropriately selected to obtain a digestion of the protein. When the enzyme to substrate ratio is unsuitably high, the correspondingly high digestion rate will not allow sufficient time for the peptides to be analyzed by mass spectrometer, and sequence coverage will be compromised. On the other hand, a low E/S ratio would need long digestion and thus long data acquisition time. The enzyme to substrate ratio can range from about 1:0.5 to about 1:200. As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

One of the widely accepted methods for digestion of proteins in a sample involves the use of proteases. Many proteases are available and each of them have their own characteristics in terms of specificity, efficiency, and optimum digestion conditions. Proteases refer to both endopeptidases and exopeptidases, as classified based on the ability of the protease to cleave at non-terminal or terminal amino acids within a peptide. Alternatively, proteases also refer to the six distinct classes—aspartic, glutamic, and metalloproteases, cysteine, serine, and threonine proteases, as classified on the mechanism of catalysis. The terms “protease” and “peptidase” are used interchangeably to refer to enzymes which hydrolyze peptide bonds.

In some exemplary embodiments, the method of detecting HCP in a sample matrix can further comprise adding protein denaturing agent to the eluate.

As used herein, “protein denaturing” can refer to a process in which the three-dimensional shape of a molecule is changed from its native state without rupture of peptide bonds. The protein denaturation can be carried out using a protein denaturing agent. Non-limiting examples of a protein denaturing agent include heat, high or low pH, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

In some exemplary aspects, the method of detecting HCP in a sample matrix can further comprise adding protein reducing agent to the eluate.

As used herein, the term “protein reducing agent” refers to the agent used for reduction of disulfide bridges in a protein. Non-limiting examples of the protein reducing agents used to reduce the protein are dithiothreitol (DTT), β-mercaptoethanol, Ellman's reagent, hydroxylamine hydrochloride, sodium cyanoborohydride, tri s(2-carboxyethyl)phosphine hydrochloride (TCEP-HCl), or combinations thereof.

In some exemplary aspects, the method of detecting HCP in a sample matrix can further comprise adding protein alkylating agent to the eluate.

As used herein, the term “protein alkylating agent” refers to the agent used for alkylation certain free amino acid residues in a protein. Non-limiting examples of the protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

In some exemplary embodiments, the digests are analyzed using a mass spectrometer.

As used herein, the term “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends heavily on the application.

In some exemplary aspects, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information, or the fragment ion signal is detectable. Tandem MS has been successfully performed with a wide variety of analyzer combinations. What analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “database” refers to bioinformatic tools which provide the possibility of searching the uninterpreted MS-MS spectra against all possible sequences in the database(s). Non-limiting examples of such tools are Mascot (http://www.matrixscience.com), Spectrum Mill (http://www.chem.agilent.com), PLGS (http://www.waters.com), PEAKS (http://www.bioinformaticssolutions.com), Proteinpilot (http://download.appliedbiosystems.com//proteinpilot), Phenyx (http://www.phenyx-ms.com), Sorcerer (http://www.sagenresearch.com), OMSSA (http://www.pubchem.ncbi.nlm.nih.gov/omssa/), X!Tandem (http://www.thegpm.org/TANDEM/), Protein Prospector (http://www.http://prospector.ucsf.edu/prospector/mshome.htm), Byonic (https://www.proteinmetrics.com/products/byonic) or Sequest (http://fields.scripps.edu/sequest).

In some exemplary aspects, the mass spectrometer can be coupled to a liquid chromatography system.

As used herein, the term “chromatography” refers to a process in which a chemical mixture carried by a liquid or gas can be separated into components as a result of differential distribution of the chemical entities as they flow around or over a stationary liquid or solid phase. Non-limiting examples of chromatography include traditional reversed-phased (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Several types of liquid chromatography can be used with the mass spectrometer, such as, rapid resolution liquid chromatography (RRLC), ultra-performance liquid chromatography (UPLC), ultra-fast liquid chromatography (UFLC) and nano liquid chromatography (nLC). For further details on chromatography method and principles, see Colin et al. (COLIN F. POOLE ET AL., LIQUID CHROMATOGRAPHY FUNDAMENTALS AND INSTRUMENTATION (2017)).

In some exemplary aspects, the mass spectrometer can be coupled to a nano liquid chromatography. In some exemplary aspects, the mobile phase used to elute the protein in liquid chromatography can be a mobile phase that can be compatible with a mass spectrometer. In some specific exemplary aspects, the mobile phase can be ammonium acetate, ammonium bicarbonate, or ammonium formate, or combinations thereof.

In some exemplary aspects, the mass spectrometer can be coupled to a liquid chromatography-multiple reaction monitoring system.

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some exemplary aspects, the mass spectrometer can be coupled to a liquid chromatography-selected reaction monitoring system.

It is understood that the present invention is not limited to any of the aforesaid host-cell protein(s), chromatographic resin(s), excipient(s), filtration method(s), hydrolyzing agent(s), protein denaturing agent(s), protein alkylating agent(s), instrument(s) used for identification, and any host-cell protein(s), chromatographic resin(s), excipient(s), filtration method(s), hydrolyzing agent(s), protein denaturing agent(s), protein alkylating agent(s), instrument(s) used for identification can be selected by any suitable means.

Various publications, including patents, patent applications, published patent applications, accession numbers, technical articles and scholarly articles are cited throughout the specification. Each of these cited references is incorporated by reference, in its entirety and for all purposes, herein.

The present invention will be more fully understood by reference to the following Examples. They should not, however, be construed as limiting the scope of the invention

### EXAMPLES

Materials and reagent preparation. WedgeWell Tris-Glycine 4-12% Mini Gels, SeeBlue Plus2 pre-stained molecular weight standards, 1M Tris-HCl (pH 8), Dynabeads MyOne Streptavidin T1 and Dynabeads Antibody Coupling Kit was purchased from Invitrogen by Thermo Fisher Scientific (Waltham, Mass.). Trans-Blot Turbo Transfer Pack was purchased from Bio-Rad (Hercules, Calif.). Formic acid, acetonitrile, Diothiothreitol (DTT) and 1-step ultra TMD-blotting solution were purchased from Thermo Fisher Scientific (Waltham, Mass.). Acetic acid, 10× Tris buffered saline (TBS), Iodoacetamide (IAM), Bovine Serum Albumin (BSA) and urea were purchased from Sigma-Aldrich (Boston, Mass.). HEPES Buffered saline with EDTA and 0.005% v/v Surfactant P-20 (HBS-EP) was purchased from GE. All monoclonal antibodies, polysorbate 20 and polysorbate 80, recombinant sialate O-acetylesterase, recombinant CHO PLBD2, anti-PLBD2 mon-clonal antibody was prepared at Regeneron Pharmaceuticals. Inc. Biotinylated Anti-CHO HCP F550 was purchased from Cygnus, Sequencing Grade Modified Trypsin was purchased from Promega (USA). Anti-SIAE monoclonal antibody was purchased from Sino Biological US Inc. Anti-Mouse IgG antibody was purchased from Abcam. Human PLBD2 was purchased from Origene Technologies Inc (Rockville, Md.). Sequencing Grade Modified Trypsin was purchased from Promega (Madison, Wis.). Anti-goat IgG antibody was purchased from Abcam (Cambridge, UK). Oasis Max column, Acquity UPLC BEH C4 column, Acquity UPLC CSH C18 column were purchased from Waters (Milford, Mass.). Acclaim PepMap 100 column and Acclaim PepMap RSLC column were purchased from Thermo Fisher Scientific (Waltham, MA). DPBS (10×) was purchased from Gibco by life technologies and Tween20 was purchased from J.T. Baker (Phillipsburg, N.J.). Q-Exactive Plus with electrospray ionization (ESI) source was purchased from Thermo Fisher Scientific (Waltham, Mass.).

Two-Dimensional Liquid Chromatography-Charged Aerosol detector (CAD)/Mass Spectrometry (MS) Assay to Detect Polysorbate Degradation. Degradation of PS20 and PS80 in CHO cell-free media or formulated antibody were analyzed by two-dimensional HPLC-CAD/MS system. The details of the setup were previously described by Genentech (Yi Li et al., Characterization and Stability Study of Polysorbate 20 in Therapeutic Monoclonal Antibody Formulation by Multidimensional Ultrahigh-Performance Liquid Chromatography-Charged Aerosol Detection-Mass Spectrometry, 86 ANALYTICAL CHEMISTRY 5150-5157 (2014)). Polysorbates were separated from formulated mAb by using Oasis MAX column (20 mm×2.1 mm, 30 μm, Waters, Milford, Mass., U.S.A.). Initial condition was set at 1% solvent B (0.1% formic acid in acetonitrile) and held for 1 min. It was increased to 20% in 1.5 minutes and dropped back down to 1% in 1.5 minutes. The up and down cycle was repeated three times until 10 minutes for complete removal of mAb from the polysorbates. By using a switch valve, Polysorbates were then subjected to separation by reversed phase chromatography using Acquity BEH C4 column (50 mm×2.1 mm, 1.7 μm, Waters, Milford, Mass., U.S.A.). Solvent B was increased to 20% from 1% from in 1.5 minutes from 10 min, then gradually increased to 99% at 45 min and held for 5 min, followed by an equilibration step of 1% B for 5 min. The flow rate was kept at 0.1 mL/min and column temperature at 40° C.

The 2D-LC system was set up with Thermo UltiMate 3000 and coupled with Corona Ultra CAD detector. Operating at nitrogen pressure of 75 psi for quantitation. Chromeleon 7 was used for system control and data analysis. Q-Exactive Plus with electrospray ionization (ESI) source was purchased from Thermo Fisher Scientific and coupled with 2DLC system for characterization only. The instrument was operated in a positive mode with capillary voltage at 3.8 kV, capillary temperature at 350° C., sheath flow rate at 40, and aux flow rate at 10. Full scan spectra were collected over the m/z range of 150-2000. Thermo Xcalibur software was used to collect and analyze MS data.

Peak area of each ester was obtained from the CAD detector and added up to account for intact PS20 or PS80. The remaining percentage of PS20 or PS80 used in this work was calculated by comparing sum of the peak area of monoester eluting between 27.5 min and 33 min at each time point to sum of peak areas at time zero. Relative percent of different order ester or total esters can be calculated similarly.

Hydrolysis of Polysorbate 20 with Sialate O-acetylesterase (SIAE) and Formulated Antibody. The effect of SIAE on PS20 was examined by mixing 16 μL 10 mM Histidine buffer, pH 6.0 and 2 μL 1% PS20, then treated with 2 μL 0.01 mg/mL, 0.025 mg/mL, 0.05 mg/mL and 0.1 mg/mL SIAE incubated at 45° C. for 5 and 10 days. One aliquot (3 μL) of each solution was diluted twenty-five time by adding 72 μL of 10 mM histidine, pH 6.0 and submitted for LC-CAD analysis. The influence of pH on the rate of PS20 degradation was determined by evaluating activity at 5.3, 6.0 and 8.0 in acetate, histidine and citrate buffer system.

The hydrolysis of PS20 in formulated mAb was examined by mixing 18 μL 75 mg/mL mAb (in original formulation or after buffer exchange to 10 mM Histidine, pH 6.0) with 2 μL 1% PS20 then incubated at 45° C. for 5 and 10 days. One aliquot (3 μL) of each solution was diluted twenty-five time by 10 mM Histidine, pH 6 and submitted for LC-CAD analysis.

Hydrolysis of Polysorbate 20 and PS80 with Putative Phospholipase B-like 2 (PLBD2) and Formulated Antibody. To evaluate the effect of PLBD2 on PS20 and PS80 degradation, 16 μL of 10 mM Histidine buffer pH 6.0 was mixed with 2 μL of 1% PS20 or 1% PS80, followed by adding 2 μL of 0.2 mg/mL PLBD2 and incubating the sample at 45° C. for 5 days. One aliquot (3 μL) of each sample was diluted twenty-five time by adding 72 μL 10 mM histidine, pH 6.0 and used for LC-CAD analysis.

The hydrolysis of PS20 in formulated mAb was examined by mixing 18 μL of 75 mg/mL mAb (buffer exchanged to 10 mM Histidine, pH 6.0) with 2 μL of 1% PS20 followed by incubation at 45° C. for 5 days. The hydrolysis of PS80 in formulated mAb was examined by mixing 18 μL of 100 mg/mL mAb (buffer exchanged to 10 mM Histidine, pH 6.0) with 2 μL of 1% PS80 followed by incubation at 45° C. for 5 days. One aliquot (3 μL) of each sample was diluted 25 times by 10 mM Histidine, pH 6 and used for LC-CAD analysis.

Detection of SIAE in CHO-Derived Antibodies (1) SIAE enrichment by immunoprecipitation: Ten mg of mAb-0 was mixed with 0, 1, 5, 10, 50, 100 μL of 0.0001 mg/mL SIAE to generate an mAb sample containing 0, 0.1, 0.5, 1, 5, 10 ppm SIAE (vs mAb-0) for creating calibration curve. Ten mg of mAb-1, mAb-2, mAb-3, mAb-4, mAb-5, mAb-6 and mAb-7 samples were also buffer exchanged to 10 mM Histidine, pH 6 for RAE measurement. 5 μL 1M acetic acid was added to each sample and incubate at room temperature for 30 minutes. 110 μL of 10× TBS and 20 μL excess 1M Trish-HCl (pH 8) were added to bring pH back to 7.5, then 25 μg of F550 biotinylated anti-HCP antibody was immediately added to each sample. Samples were incubated with gentle rocking at 4° C. overnight. 1.5 mg magnetic beads were added to each sample after being washed and suspend in 1× TBS and incubate at room temperature with gentle rotating for 2 hours. Beads were then washed by HBS-T and 1× TBS and elute by 100 μL of 50% acetonitrile, 0.1M acetic acid in MilliQ water by shaking at 800 rpm for 5 minutes twice. Each antibody sample was dried and resuspended in 20 μL urea denaturing and reducing solution (8M urea, 10 mM DTT, 0.1M Tris-HCl pH 7.5), incubated at 500 rpm at 56° C. for 30 minutes. Six μL of 50 mM iodoacetamide was then added to each sample to mix and react at room temperature in the dark for 30 minutes. 50 μL of 20 ng/μL trypsin was added to each sample for digestion at 37° C., sharking at 750 rpm overnight. The digested samples were acidified by 4 μL 10% formic acid and 20 μL were transferred to glass vials for LC-MS/MS analysis and the rest were stored at −80° C.

(2) LC-multiple reaction monitoring (MRM) quantitation of SIAE: The SIAE enriched digested samples were subjected to LC-MRM analysis. LC-MRM analysis was performed on an Agilent 6495A QQQ Mass Spectrometry (Agilent, Wilmington, Del.) equipped with an Agilent 1290 infinity HPLC (Agilent, Wilmington, Del.). 15 μL of the digested samples were injected onto an Acquity CSH C18 column (50 mm×2.1 mm, 1.7 μm, Waters, Milford, Mass., U.S.A) at 60° C. using 0.1% formic acid in water as mobile phase A, and 0.1% formic acid in acetonitrile as mobile phase B. The column was equilibrated at 10% B mobile phase B for 2 min, linearly increased to 50% in 8 minutes and then increased to 90% and kept for 3 min, then re-equilibrated at 10% mobile phase B for 2 min. Elution was performed at 0.4 mL/min and peaks between 2-13 min analyzed using an ESI source operating at positive mode, with gas temperature 200° C., gas flow 12 L/min, nebulizer gas 20 psi, sheath gas temperature 300° C., sheath gas flow 11 L/min, capillary voltage 3500V and nozzle voltage 500 V. SIAE were monitored at 540.80/864.42 (LLSLTYDQK (SEQ ID NO.: 1)) for quantitation and 639.35/865.45 (ELAVAAAYQSVR (SEQ ID NO.: 2)) for confirmation. Peak integration was performed by Skyline, and SIAE concentrations were calculated based on the calibration curved created by spiked-in SIAE.

Western Blot of SIAE. Samples were prepared by mixing 5 μL (0.002 mg/mL, 0.01 mg/mL and 0.02 mg/mL) SIAE, 5 μL 0.25 M IAM and 10 μL 2× Tris-Glycine loading buffer, heating at 80° C. for 2 min and then loaded onto SDS-PAGE gel, electrophorese at 160V for 1.5 hours and then transfer to PVDF membrane at 25V for 30 minutes. The PVDF membrane was then blotted by 2% BSA in PBST for 1 hour at room temperature, followed by adding anti-SIAE monoclonal antibody in 1% BSA (1:1000) and incubating at 4° C. overnight. After washed with PBST three times, the secondary antibody anti-mouse IgG was added at 1:5000 at room temperature for 1 hour. PVDF was then washed by PBST three times and stained by 1-step ultra TMD-blotting solution.

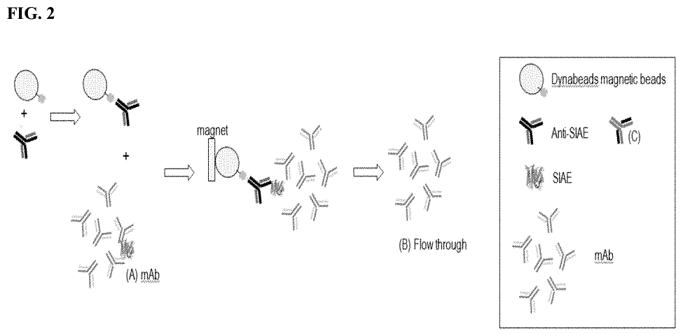

Depletion of SIAE from CHO-Derived Antibodies. SIAE depletion experiment was performed by using Dynabeads Antibody Coupling Kit (See FIG. 2). Five mg magnetic Dynabeads were first mixed with 100 ug anti-SIAE in C1 and C2 buffer from the kit, and then incubated by gentle rocking at 4° C. overnight. Beads were washed by HB, LB and SB from the kit and then resuspended into 500 μL water. 50 μL resuspended anti-SAIE Dynabeads were added to each 10 mg mAb samples to a total volume of 500 μL, rotating 2 hrs at room temperature. Supernatant was removed and dried under SpeedVac and resuspended into water. Protein concentration of mAb was measured and adjusted t0 75 mg/mL for incubation with 0.1% PS20. Five mg magnetic Dynabeads were mixed with 100 μg irrelevant antibody and went through same process as negative control.

LC-multiple reaction monitoring (MRM) quantitation of PLBD2. Antibody mAb-8 is an IgG4 antibody expressed from a control cell line without knocking out any genes; mAb-9 is the same IgG4 antibody as mAb-8 but expressed from a cell line with PLBD2 gene knocked out; mAb-10 is mAb-8 being further purified by the step to remove PLBD2; mAb-11, mAb-12, mAb-13 and mAb-14 are different IgG4 antibodies without PLBD2 removal purification step; mAb-15 is an IgG1 antibody without PLBD2 removal step.

Both purified antibody mAb-10 with spiked-in PLBD2 standard and mAb drug substance (mAb-10, mAb-11, mAb-12, mAb-13, mAb-14, mAb-15) were digested by trypsin and then were subjected to LC-MRM analysis. LC-MRM analysis was performed on an Agilent 6495A QQQ Mass Spectrometry (Wilmington, Del.) equipped with an Agilent 1290 infinity HPLC (Wilmington, Del.). 20 μL of the digested samples were injected onto an Acquity BEH C18 column (2.1×50 mm, 1.7 μm) at 40° C. pre-equilibrated with 88% mobile phase A (0.1% formic acid in water) and 12% mobile phase B (0.1% formic acid in acetonitrile) at a flow rate of 0.4 mL/min. Post sample injection the gradient was maintained isocratically at 12% B for 0.5 min, followed by a linear increase to 15% B over 6 minutes and then increased to 90% B in 0.1 min after which the gradient was kept at 90% B for 2.5 minutes. In the end, the gradient was decreased to 12% B to allow the column to be re-equilibrated for 3 minutes. Eluent between 2-13 minutes was analyzed using an ESI source operating under positive mode, with gas temperature of 250° C., gas flow of 12 L/min, nebulizer gas of 20 psi, sheath of gas temperature of 300° C., sheath gas flow of 11 L/min, capillary voltage of 3500V and nozzle voltage of 500 V. PLBD2 were monitored at 615.35/817.41 (SVLLDAASGQLR (SEQ ID NO.: 4)) for quantitation and 427.7/450.3 (YQLQFR (SEQ ID NO.: 3)) for confirmation. Peak integration was performed by Skyline (Brendan Maclean et al., Skyline: an open source document editor for creating and analyzing targeted proteomics experiments, 26 BIOINFORMATICS 966-968 (2010)), and PLBD2 concentrations were calculated based on the calibration curved created by spiked-in PLBD2 standards.

Western Blot of PLBD2. Western blot was performed to confirm the existence of PLBD2. Samples were prepared by mixing 12.5 μL mAb-8 (4 mg/mL) with 2.5 μL 0.25 M IAM and 10 μL 2× Tris-Glycine loading buffer, followed by heating at 80° C. for 2 minutes. 20 μL sample was loaded onto the SDS-PAGE gel for electrophoresis separation at 160V for 1.5 hours, and the separated proteins were transferred to PVDF membrane at 25V for 30 minutes. The PVDF membrane was then blotted by 2% BSA in PBST for 1 hour at room temperature, followed by adding anti-PLBD2 monoclonal antibody in 1% BSA (1:1000) and incubating at 4° C. overnight. After washing with PBST three times, the secondary antibody anti-goat IgG was added at 1:5000 at room temperature for 1 hour. The PVDF membrane was then washed by PBST three times and stained by 1-step ultra TMD-blotting solution.

Depletion of PLBD2 from CHO-Derived Antibodies. PLBD2 depletion experiment was performed by using Dynabeads antibody coupling kit. Five mg magnetic Dynabeads were first mixed with 100 μg anti-PLBD2 mAb in C1 and C2 buffer from the kit, and then incubated by gentle rocking at 4° C. overnight. Beads were washed by HB, LB and SB from the kit and then resuspended into 500 μL water. 50 μL resuspended anti-PLBD2 Dynabeads were added to each 10 mg mAb samples to a total volume of 500 μL respectively, followed by shaking at room temperature for 3 hours. After removing the beads, the supernatant was dried under SpeedVac and resuspended into water. Protein concentration of mAb was measured and adjusted to 75 mg/mL for incubation with 0.1% PS20.

Shotgun proteomics analysis of PLBD2. Both commercial and in-house-made PLBD2 were subjected to shotgun proteomics analysis. 10 μg of PLBD2 was dried with Speedvac, then reconstituted with 20 μl of denature/reduction buffer containing 8M urea and 10 mM DTT. The proteins were denatured and reduced at 37° C. for 30 minutes, and then incubated with 6 μl of 50 mg/ml iodoacetamide for 30 minutes in dark. Alkylated proteins were digested overnight with 50 μl 0.01 μg/μL trypsin at 37° C. The peptide mixture was acidified by 5 μL of 10% TFA. The sample was injected 10 μL for LC-MS/MS analysis.

PLBD2 knockout cell line generation. In order to target PLBD2 for disruption using CRISPR/Cas9, a small guide RNA (sgRNA) sequence corresponding to Exon 1 of PLBD2 was selected for specific targeting of PLBD2 exons 1. Sense (5′-TGTATGAGACCACGCCCCCATGGACCGGAGCCC-3′) (SEQ ID NO.: 10) and antisense (5′-AAACGGGCTCCGGTCCATGGGGCGTGGTCTCA-3′) (SEQ ID NO.: 11) oligonucleotides were ordered, with appropriate overhangs for cloning into CAS940A-1 (System Biosciences). The paired oligonucleotides were annealed at 5 μM by incubation at 95° C. for 5 min followed by cooling to room temperature gradually. The annealed oligos were diluted 10× in water and ligated into CAS940A-1 using T4 DNA ligase (ThermoFisher Scientific, Waltham, Mass.). After transformation of Electromax DH10B cells (ThermoFisher Scientific, Waltham, Mass.), colonies were screened by sequencing. Maxi-preps of sequence verified plasmids containing PLBD2 sgRNA 1 was generated using the EndoFree Plasmid Maxi Kit (Qiagen).

### Example 1

### Polysorbate in mAb Formulation Detected by 2D-LC-CAD/MS

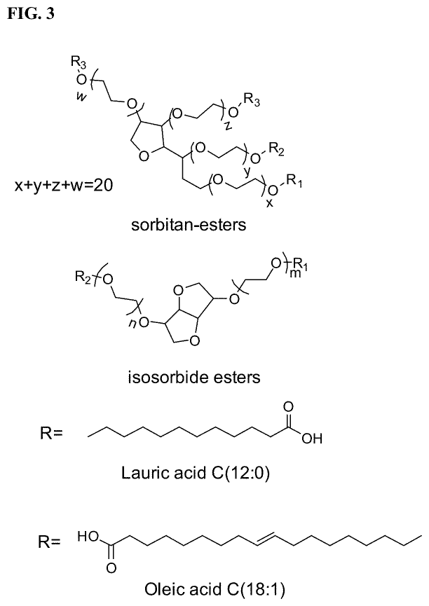

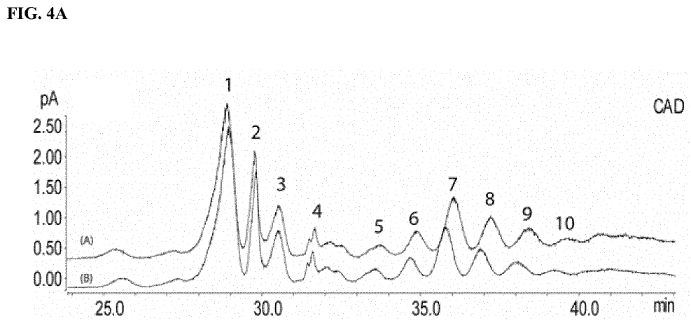

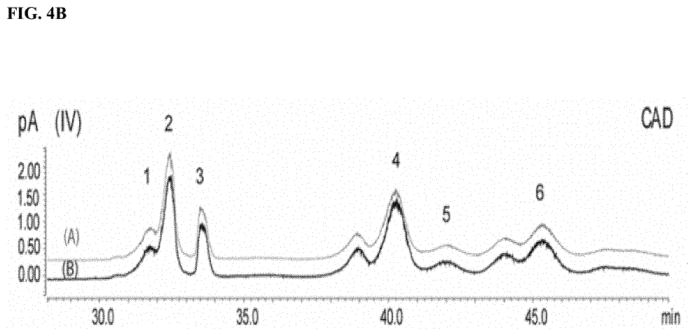

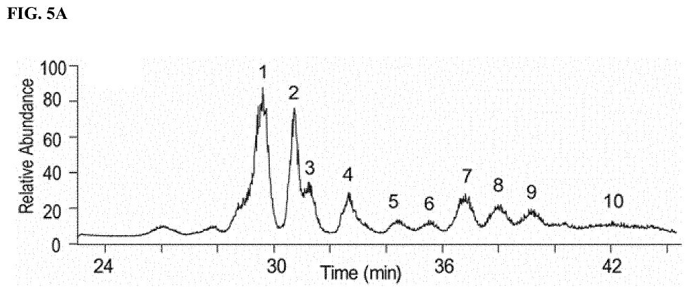

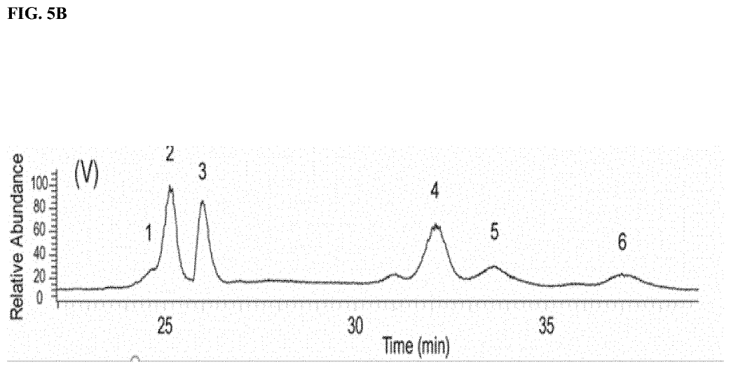

Polysorbate in formulated mAbs was detected and identified by 2D-LC-CAD/MS following slightly modified method by Yi Li et al., supra. Since changes after hydrolysis occurs on ester bonds in the above study, the gradient was set to remove most of POE, POE sorbitan, POE isosorbide, as well as mAb by Oasis Max column, leaving mainly all forms of POE esters. Reverse phase chromatography was then used to separate the remaining POE esters based on their fatty acid content and type. The esters eluted in the order of monoesters, diesters, triesters and tetraesters (FIG. 3). The structure of each ester was elucidated by mass spectrometry based on the chemical formula of the polymer and dioxolanylium ion generated by in source fragmentation, FIG. 5A and FIG. 5B show the representative total ion current (TIC) profile of PS20 and PS80 with major peaks labeled. Quantitation of polysorbates was determined by Charged Aerosol Detector (CAD). FIG. 4A and FIG. 4B show the recovery of PS20 in PS20 standard solution and a formulated mAb by using 2D-LC/CAD and PS80 in PS80 standard solution and a formulated mAb by using 2D-LC/CAD, respectively. For FIGS. 4A-B, the corresponding peaks were identified by mass spectrometry.

### Example 2

### SIAE in Formulated mAb with Polysorbate 20 Degradation

Owing to their low level, identification and quantitation of host cell proteins swamped in highly concentrated drug product can be often analytically challenging. Immunoprecipitation was used to enrich HCPs using CHO HCP ELISA kit F550 (Cygnus, Southport, N.C.). Several mAb samples were subjected to shotgun proteomic analysis after enrichment by immunoprecipitation (data not shown) and approximately 30 HCPs were identified with high confidence. After excluding proteins that are apparently not enzymatic active or not targeting ester bond, for examples, C-C motif chemokine, complement C3 and sulfhydryl oxidase, a number of proteins were selected to be overexpressed and purified in CHO. These recombinant proteins were then subject to PS20 degradation activity assay by incubating with 0.1% PS20 at 45° C. Among these proteins, Sialate-O-acetylase (SIAE) was frequently identified in the drug substances which showed strong PS20 degrading activity. The SIAE degradation pattern was further examined.

### Example 3

### Degradation Pattern in Formulated mAb and with Recombinant Sialate O-Acetylesterase (SIAE)

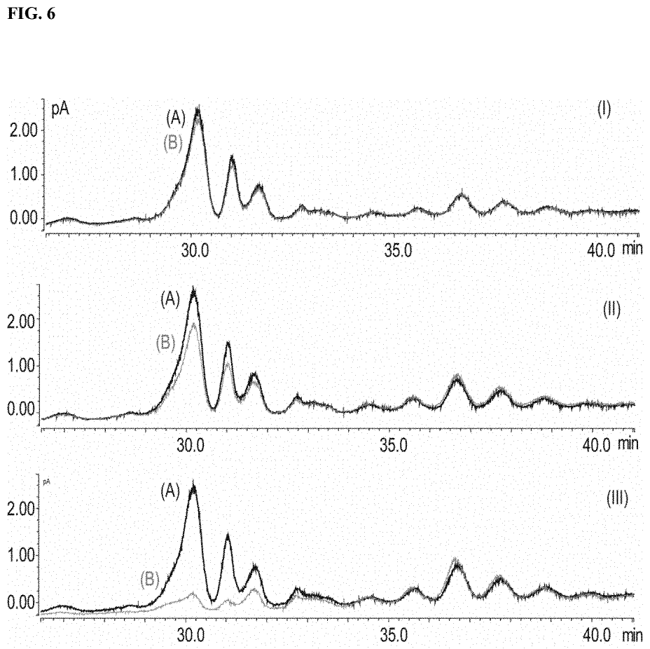

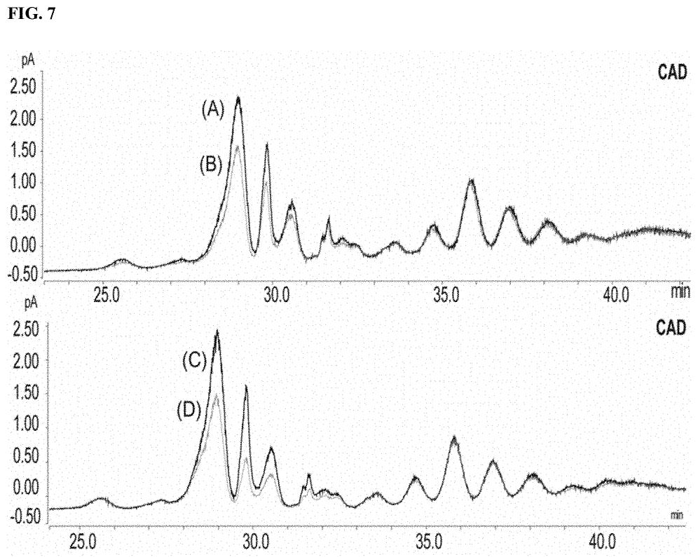

Polysorbate 20 degradation induced by recombinant SIAE was monitored. Recombinant SIAE with concentration ranging from 1 to 10 ppm was incubated with 0.1% of PS20 for 0, 5 and 10 days (FIG. 6, day 5 data not shown). Significant PS20 degradation was observed when SIAE concentration was 2.5 ppm with 10-day incubation. A closer look at the chromatograph of PS20 showed that the decreases were occurred only on those peaks eluting at early times between 27.5 and 33 min. These POE esters are monoester containing short fatty acid chain, including POE sorbitan monolaurate, POE isosorbide monolaurate, POE sorbitan monomyristate and POE isosorbide monomyristate. The POE esters with longer chains, including POE isosorbide monopalmitate, POE isosorbide monosterate and POE sorbitan with higher order esters, however, did not show noticeable change during incubation, indicating that the enzyme preferentially targeted the short chain fatty acid monoester. This degradation pattern had not been reported in other previous lipase studies of manufactured mAbs, (Dixit et al, supra; Chiu et al, supra; Hall et al, supra, Labrenz, supra). However, a recent investigation conducted by Mcshan et al. demonstrated a similar pattern when incubating PS20 with a carboxylic ester hydrolase, purified pancreatic lipase type II (McShan et al, supra). According to GO ancestor chart, sialate O-acetylesterase activity (GO:0001681) could trace back to short chain carboxylic ester hydrolase activity (GO: 0034338), which agrees with the observed unique PS20 degradation pattern with short-chain preferred cleavage. Since SIAE has been frequently detected in formulated mAbs, the PS20 degradation pattern for those mAb samples containing SIAE was also examined. Representative chromatogram of the time course experiment for PS20 degradation of a formulated mAb (FIG. 6, bottom panel) is shown together with that of recombinant SIAE (FIG. 7, upper panel). The two traces of chromatograph are almost identical, suggesting that SIAE could be the potential root cause for PS20 hydrolysis.

### Example 4

### Effect of pH on Degradation of PS20 by SIAE

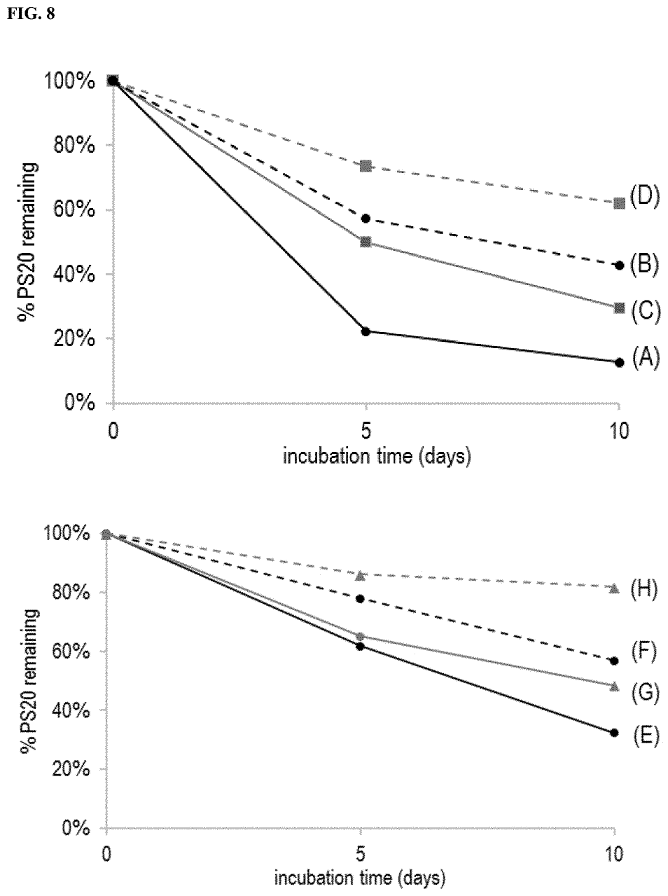

Three typical formulation buffers with different pH values were tested to evaluate the pH effects on PS20 degradation. The three buffer solutions were citrate buffer for pH 8.0, histidine buffer for pH 6.0 and arginine hydrochloride buffer for pH 5.3 (FIG. 8). Higher percentage of PS20 loss was observed at pH 6.0 compared to pH 8.0 and 5.3 for mAb-1 and mAb-2 incubating with PS20, respectively. Similar response to pH was observed when SIAE directly incubated with PS20 in buffers with different pH, also with maximum degradation at pH 6.0.

### Example 5

### Correlation of Amount of SIAE in Formulated mAb to PS20 Loss Over Time

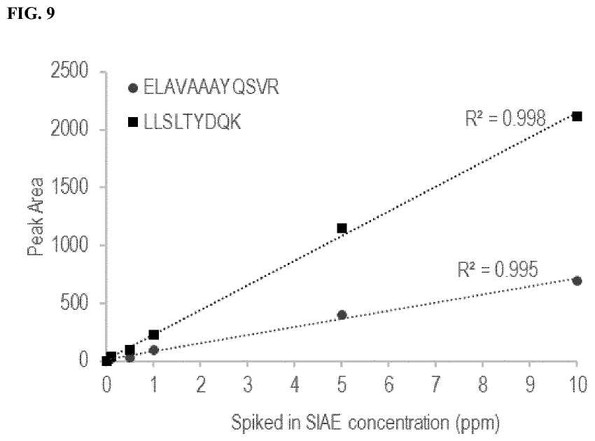

SIAE has been shown to hydrolyze PS20, however, other lipase(s)/esterase(s) may also participate this degradation process. To rule out the possibility of other esterase-like enzymes' participation, it was necessary to establish a correlation between enzymatic activity and endogenous SIAE amounts. The rationale was that for certain mAb samples with SIAE-type hydrolyzing pattern, if SIAE amount is positively correlated with its lipase activity, most likely it is the only enzyme responsible for PS20 hydrolysis. Otherwise, there should be some other enzymes involved. Two SIAE peptides (LLSLTYDQK (SEQ ID NO.: 1) [3.2 min] and ELAVAAAYQSVR (SEQ ID NO.: 2) [3.6 min]) were chosen to quantitate SIAE in formulated mAb using multiple reaction monitoring technology (MRM). SIAE spiked-in mAb was used to create calibration curve. Standard curves (0.1-10 ppm) with coefficients 0.998 and 0.995 were generated for each of the peptide (FIG. 9), concentration of SIAE in each sample was then obtained by extrapolating it peak area onto the curve. In total, 10 mAbs were subjected to SIAE quantitation. Quantitative examination of peak area of these ten mAbs determined the concentration of SIAE in the formulated mAb were between 0.2 to 4 ppm per mg mAb.

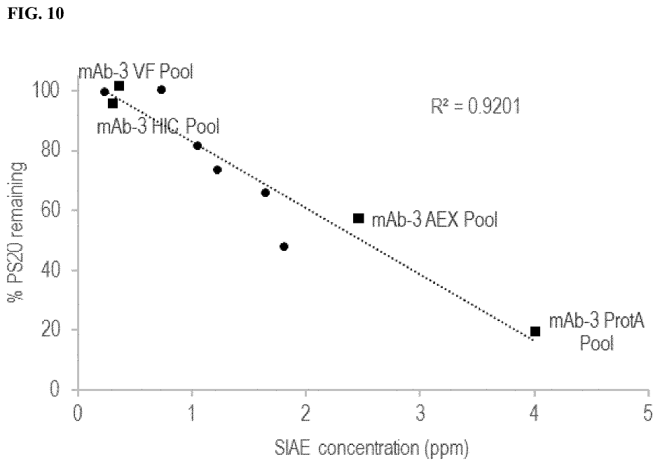

PS20 degradation was measured for the same 10 mAbs after concentrated and buffer exchanged to 10 mM Histidine buffer, pH 6. The percentage of the remaining PS20 was plotted against SIAE concentration and correlation coefficient R² was calculated to evaluate the linear dependence of the two variables (FIG. 10). A downhill linear relationship with calculated Pearson correlation coefficient of 0.92 indicates a strong negative correlation between these two variables, suggesting SIAE concentration in drug substances is positively correlated to the PS20 loss during incubation. Among the ten mAb samples, four of them were in-process samples (mAb3) from four consecutive processing steps, which are Protein A, AEX, HIC and VF pool, respectively (filled square markers in FIG. 10). Protein A was the first major step used to remove most HCPs. After this step, SIAE concentration remained to be as high as 4 ppm, resulting in a high enzymatic activity with only as 22% of PS20 remaining after 5 days. When anion ion exchange (AEX) was applied, SIAE concentration was decreased to 2.4 ppm with higher than 60% of PS20 remaining. Further refinement of the washing by HIC and VF removed more SIAE, leaving less than 0.3 ppm of SIAE and almost all PS20 was preserved in the solution. These four samples were perfectly positioned on the linear regression line, indicating SIAE played a key role in PS20 degradation.

### Example 6

### Depletion of SIAE Results in Decreased Level of PS20 Degradation

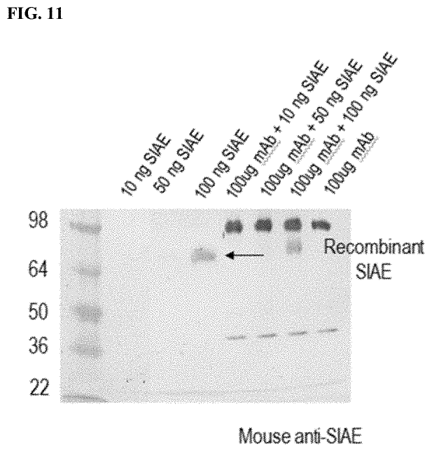

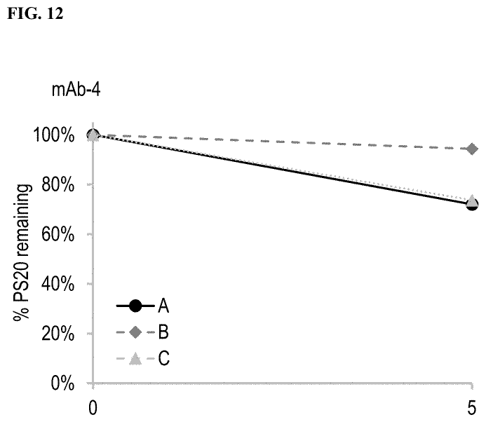

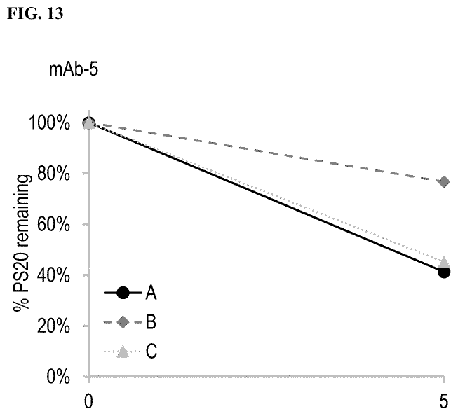

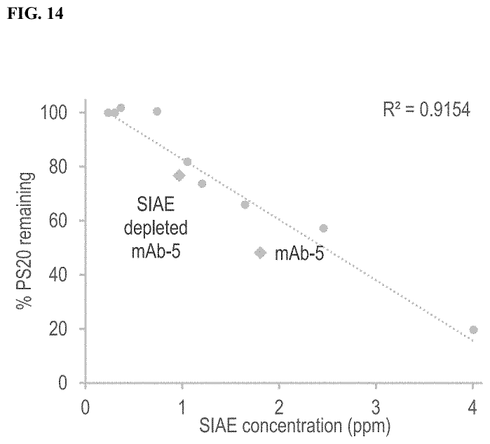

To further examine whether PS20 degradation was solely caused by the presence of SIAE in those formulated mAbs, RAE depletion experiment was performed. The rationale was that if PS20 degradation is caused by SIAE, but not any other HCPs, depletion will result in a diminished PS20 degradation and the degradation degree will depend on how much SIAE has been removed. FIG. 2 showed the depletion scheme for mAbs. Human anti-SIAE antibody was covalently coupled to Dynabeads for depletion of SIAE. One irrelevant antibody was also covalently coupled to Dynabeads and served as the negative control. First, it was validated that anti-SIAE was able to bind specifically to SIAE by Western blot. As shown in FIG. 11, Western blot can detect SIAE when 100 ng was loaded with or without mAb present. It should be noted that when loading below 100 ng, for example, with 10 ng and 50 ng of SIAE loading, this antibody failed to detect any SIAE. The antibody used in this experiment was against human SIAE. Given only around 70% of sequence homology, it was not surprising that this antibody did not bind to CHO-SIAE with very high affinity. Therefore, when large amount of SIAE was present in the sample, it may not be able to fully remove them. For mAb-4, before SIAE-depletion, SIAE was active to degrade approximately 26% of PS20 at 45° C. for 5 days. After SIAE-depletion, the esterase activity was measured to be negligible, indicating -SIAE was the root cause of PS20 degradation in mAb-4. This removal was specific for anti-SIAE antibody as the negative control, an irrelevant antibody, did not change the esterase activity at all (FIG. 12). However, for mAb-5, although significant reduction of esterase activity was observed (41%-77% of remaining PS20), about 23% of PS20 loss was still found after depletion (FIG. 13). This remaining activity was not surprising since anti-SIAE non-CHO antibody with low affinity was used to SIAE in mAbs. It was likely that the depletion was not complete, leaving trace amount of SIAE in mAb solutions. To confirm that residue SIAE present in the remaining solution after depletion, IP-MRM-MS was performed on the sample. The IP-MRM-MS results were added to the previous ten data sets as marked with filled diamonds in FIG. 14. Before depletion, the concentration was 1.8 ppm and it was reduced to 0.97 ppm after depletion. The remaining SIAE fitted to the Pearson correlation curve perfectly, suggesting it was the remaining SIAE while not other HCP responsible for PS20 degradation.

Each polysorbate component can be hydrolyzed with different efficiency, therefore, the PS20 degradation pattern observed in the formulated mAbs can be used as the fingerprint to identify and verify the enzymes responsible for PS20 hydrolysis. The PS20 degradation profile observed in the formulated mAbs studied in this work demonstrates specific cleavage of monoesters rather than higher order esters. The esterase activity was also prone to the monoesters containing tail groups (fatty acids) with shorter chain length (C12, C14).

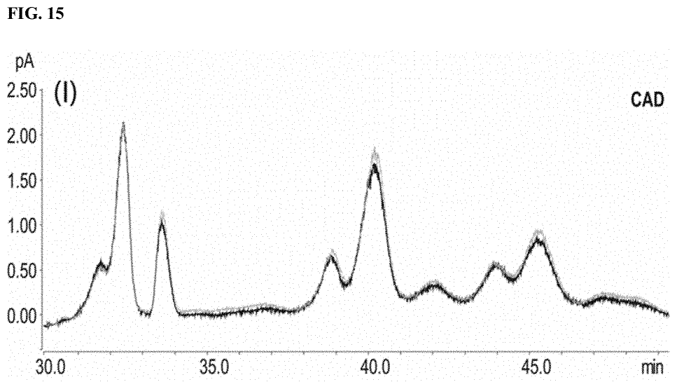

It was noted that the esterase activity of SIAE was specific to PS20 only but not to PS80. Structurally, PS80 is different from PS20 which contains monoesters with unsaturated long chain fatty acid (C18:1/C18:2) and higher-order esters (See FIG. 3). SIAE prefers cleaving the site on the monoesters with short fatty acid chains. As shown in FIG. 15, PS80 did not show any degradation when incubated with SIAE for 5 days at 45° C. Further structural and biochemical studies may be useful to understand the unique cleavage pattern of SIAE, which could be related to the bulkiness of hydrophobic POE ester moieties. The specific esterase activity of SIAE on PS20 do suggest a possible advantage of using PS80 over PS20, although oxidation may be an issue to be considered when using PS80 (Oleg V. Borisov, Junyan A. Ji & Y. John Wang, Oxidative Degradation of Polysorbate Surfactants Studied by Liquid Chromatography-Mass Spectrometry, 104 JOURNAL OF PHARMACEUTICAL SCIENCES 1005-1018 (2015); Erlend Hvattum et al., Characterization of polysorbate 80 with liquid chromatography mass spectrometry and nuclear magnetic resonance spectroscopy: Specific determination of oxidation products of thermally oxidized polysorbate 80, 62 JOURNAL OF PHARMACEUTICAL AND BIOMEDICAL ANALYSIS 7-16 (2012)).

As seen in FIG. 9, since there is a positive correlation between SIAE concentration and PS20 degradation, use of CHO-SIAE knockout cell lines can eliminate SIAE expression and can therefore reduce polysorbate degradation while maintaining the routine purification process.

### Example 7

### LAL and SIAE in Formulated mAb with Polysorbate 20 Degradation

In the test carried out as shown in Example 1, another protein lysosomal acid lipase (LAL) was also identified in the drug substances which showed PS20 degrading activity. LAL can hydrolyze ester bonds at both the primary and secondary esters and for all fatty acids.

To examine the effect of both SIAE and LAL on PS20, 10 ppm of LAL and 10 ppm of SIAE were incubated with a solution comprising 0.2% PS20. Polysorbate in formulated mAbs were detected and identified as shown in Example 1.

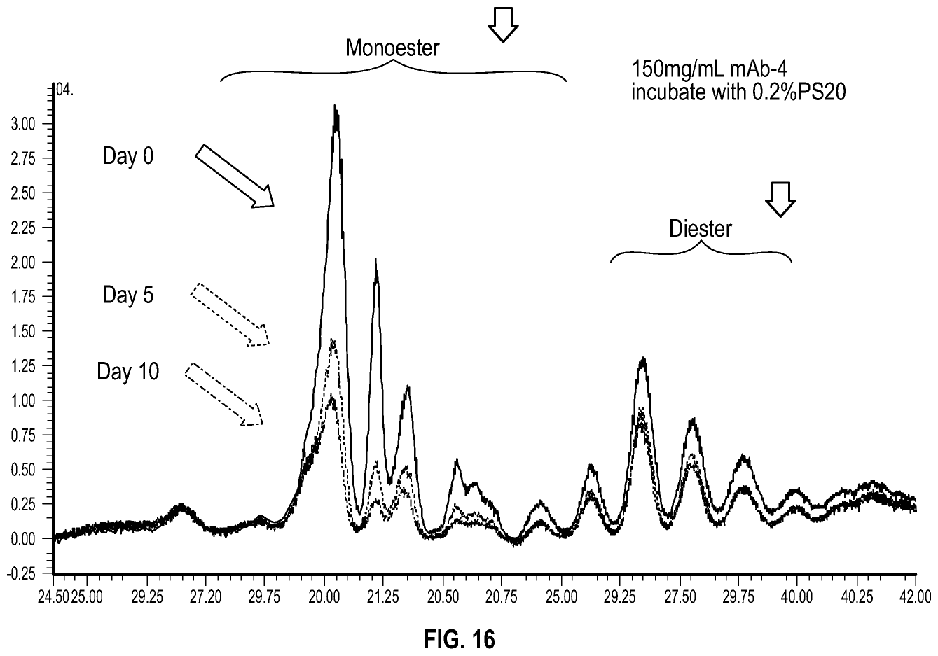

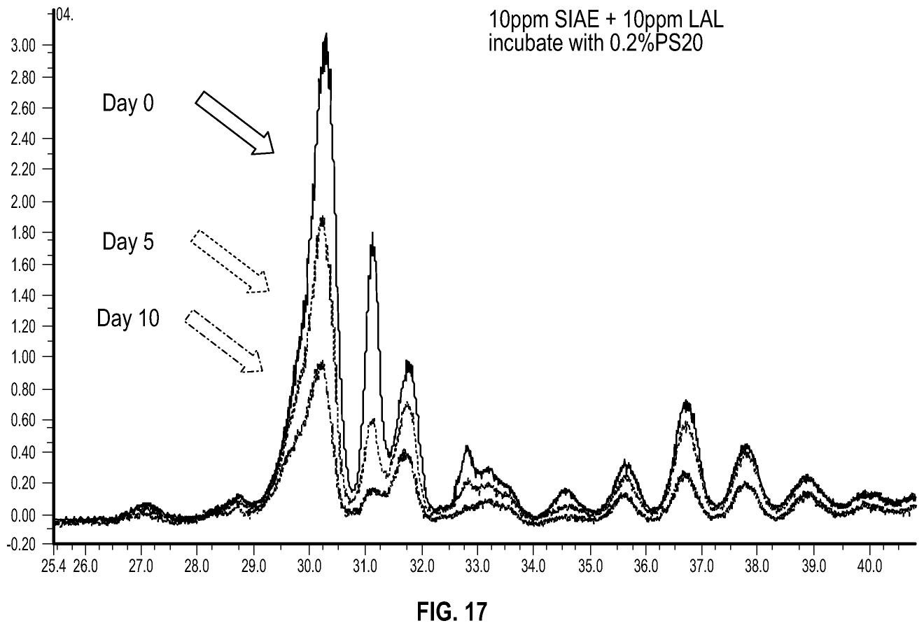

FIG. 16 shows the representative total ion CAD profile of PS20 in a formulation with mAb-4 with major peaks labeled, containing sorbitan monoester, isosorbide monoester and diesters with a variety of fatty acid chains. Comparison of this profile with the profile of 0.2% PS20 incubated with 10 ppm LAL and 10 ppm SIAE (FIG. 17) shows that both LAL and SIAE can contribute towards PS20 degradation. For both the experiments, all ester species degraded after 5 days.

### Example 8

### LAL in Formulated mAb with Polysorbate 20 Degradation

To further examine the effect of presence of LAL in a formulated mAb, LAL depletion experiment was performed. The rationale was that if PS20 degradation was caused by LAL, but not any other HCPs, depletion will result in a diminished PS20 degradation and the degradation degree will depend on how much LAL has been removed.

The LAL depletion scheme, similar to the SIAE depletion scheme (as shown in FIG. 2) was performed.

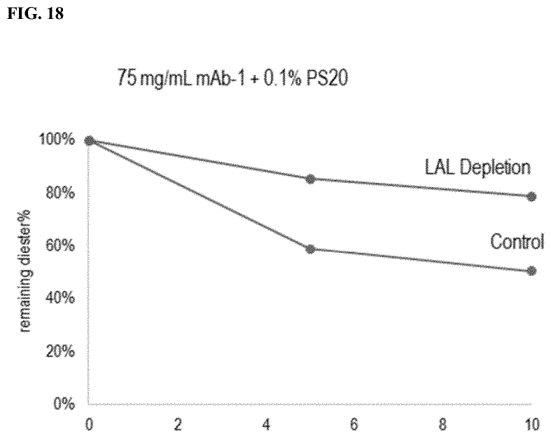

Human anti-LAL antibody was covalently coupled to Dynabeads for depletion of LAL. One irrelevant antibody was also covalently coupled to Dynabeads and served as the negative control. First, it was validated that anti-LAL was able to bind specifically to LAL by Western blot (not shown). For mAb-1, before LAL-depletion, LAL was active to degrade approximately 50% of diesters in PS20 at 45° C. in 10 days (FIG. 18). After LAL-depletion, the diesters in PS20 degradation was approximately 20% indicating that LAL can be a potential cause of PS20 degradation in mAb-1.

### Example 9

### PS20 Degradation in Formulated mAb Using LIPA Knockout

In order to target LAL for disruption using CRISPR/Cas9, two guide RNA sequences were designed for specific targeting of LIPA exons 2 and 3. Both guides were cloned into a sgRNA expression plasmid which contains elements for site-specific integration into CHO cells. The sgRNA expression plasmid contains two minimal human H1 promoters driving expression of a guide RNA and the tracrRNA following the sgRNA. For site-specific stabilization, the sgRNA expression plasmid was co-stabilized at EESYR with a second plasmid that transcribes the spCas9 nuclease. After transfection and about ten days of Hygromycin B selection, the observable recombinant pool was sorted. The disruption of LAL in the sorted pool was confirmed by gDNA qPCR, cDNA qPCR and trypsin digest mass spectrometry. The targeting guide sequences for the LIPA knockout were 5′-GTACTGGGGATACCCGAGTG-3′ (SEQ ID NO.; 8) (nucleotides 120-139, sense strand) and 5′-CCAGTTGTCTATCTTCAGCA-3′ (SEQ ID NO.: 9) (nucleotides 232-251, sense strand).

LAL depletion in a formulation for mAb-1 (See FIG. 18) decreased PS20 degradation on LAL depletion.

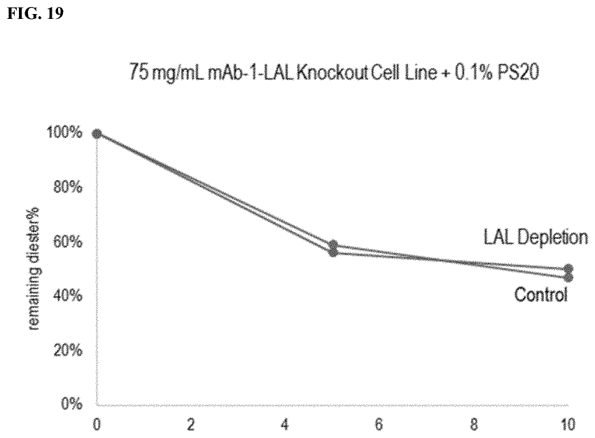

Further, a complete absence by LIPA knockout did not decrease PS20 degradation. In this experiment, the mAb-1 prepared using the CHO-LIPA knockout cells, approximately 50% of PS20 was degraded in ten days (See FIG. 19), which was similar to the effect seen for mAb-1 prepared using CHO cells with regular LAL expression levels.

### Example 10

### Polysorbate 80 Degradation by LAL

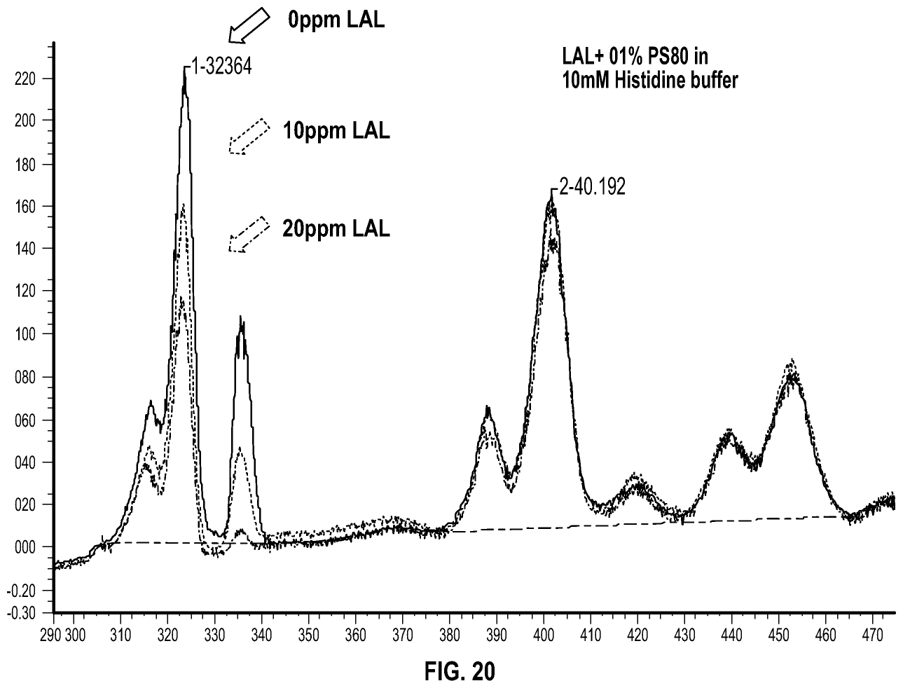

LAL has an ability to hydrolyze primary and higher order esters. As shown in FIG. 20, PS80 showed a significant degradation of monoesters only when incubated with LAL at a concentration of 10 ppm and 20 ppm at 5 days at 45° C.

### Example 11

### Polysorbate 80 Degradation by LAL in Formulated Products

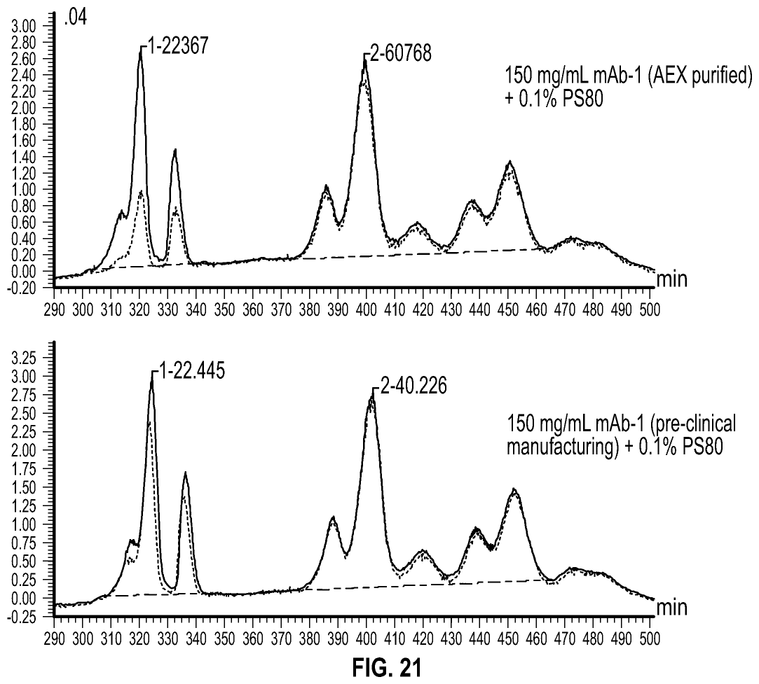

Formulated mAb-1 obtained from different programs were evaluated for its PS80 degradation profile. For mAb-1 (AEX purified) and mAb-1 (pre-clinical manufacturing) incubated with 0.1% PS80, the degradation profile at 5 days on incubation at 45° C. is shown in FIG. 21.

### Example 12

### PS20 Degradation in Formulated mAb Using LIPA Knockout

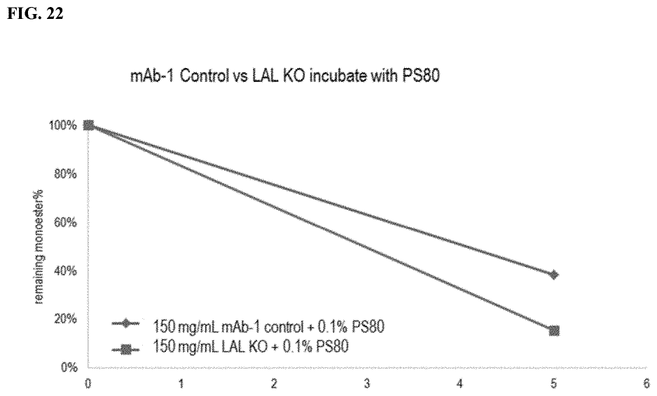

For mAb-1 prepared using CHO cells with regular LAL expression levels, PS80 degradation was observed to be 60% (FIG. 22), whereas for mAb-1 prepared using the CHO-LIPA knockout cells, PS80 degradation was observed to be approximately 85%. The mAb-1 prepared using the CHO-LIPA knockout cells showed a higher degradation.

Similar to the comparison of complete absence of LAL in the formulation with PS20, a complete absence of LAL did not show a significant decrease in PS80 degradation profile, which could be due to an increase in expression of other unidentified lipase(s), which have similar activity as LAL in degrading PS80.

Thus, the observation of free fatty acid particulates along with degradation of PS20 and PS80 in formulated monoclonal antibodies led to the identification of sialate-O-acetylesterase and lysosomal acid lipase as host-cell proteins responsible for free fatty acid particulates along with degradation of PS20 and PS80.

Recombinant SIAE was obtained by overexpressing in CHO cell and characterized its enzymatic activity. SIAE demonstrated strong hydrolysis activity for PS20 at low ppm level with unique pattern. SIAE was detected and quantitated in multiple formulated mAbs and amount of SIAE is correlated with PS20 loss. When SIAE was depleted from mAbs, the hydrolysis is also diminished. The studies show low levels of SIAE presented in formulated mAb plays the key role in the degradation of PS20 in some antibody formulations. SIAE prefers cleaving the site on the monoesters with short fatty acid chains. As shown in FIG. 15, PS80 did not show any degradation when incubated with SIAE for 5 days at 45° C. due to the unique cleavage pattern of SIAE.

Similarly, recombinant LAL was obtained by overexpressing in CHO cell and characterized its enzymatic activity. LAL demonstrated a hydrolysis activity for PS20 with a unique pattern. When LAL was depleted from mAbs, the hydrolysis of higher order esters of PS20 was also diminished. LAL also demonstrated a hydrolysis activity for PS80. However, a complete absence of LAL did not show a decrease in the hydrolysis of either PS20 or PS80 which could be attributed to the upregulation of other lipase(s) which compensate for the absence of LAL.

### Example 13

### PLBD2 in Drug Substance

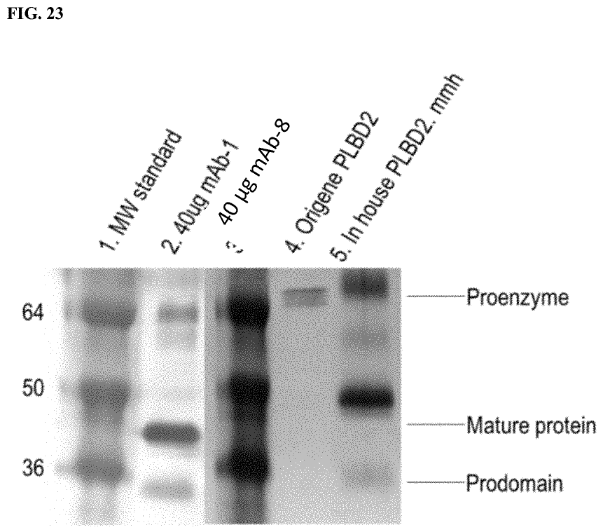

It has been reported that PLBD2 proenzyme (MW 64 kDa) was able to undergo limited autolysis leading to the formation of a 28 kDa N-terminal prodomain and a 40 kDa C-terminal mature protein (Florian Deuschl et al., Molecular characterization of the hypothetical 66.3-kDa protein in mouse: Lysosomal targeting, glycosylation, processing and tissue distribution, 580 FEBS LETTERS 5747-5752 (2006); Kristina Lakomek et al., Initial insight into the function of the lysosomal 66.3 kDa protein from mouse by means of X-ray crystallography, 9 BMC STRUCTURAL BIOLOGY 56 (2009)). Three different forms of PLBD2 at MW of 64 kDa, 40 kDa and 28 kDa were all observed on western blot of the drug substance mAb-8 (FIG. 23, lane 2). CHO PLBD2 expressed in-house contained all three different forms of PLBD2 (FIG. 23, lane 5). Interestingly, recombinant human PLBD2 purchased from OriGene contained only proenzyme at 66 kDa (FIG. 23, lane 4).

### Example 14

### PS20 and PS80 Degradation Pattern with Human PLBD2 and CHO PLBD2

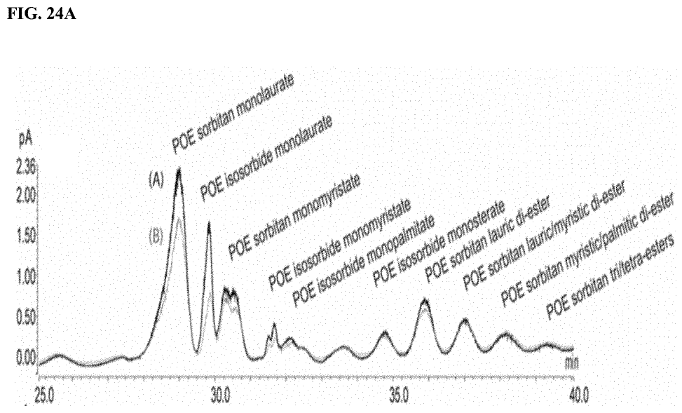

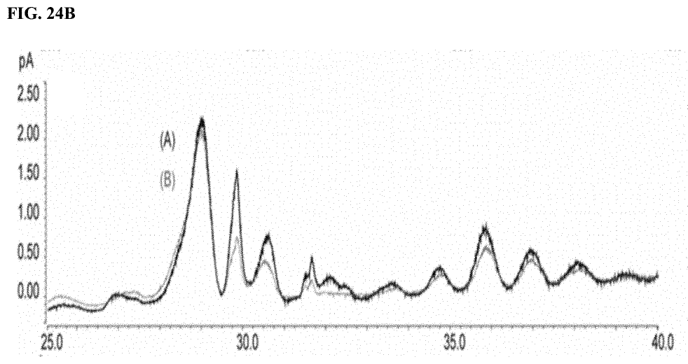

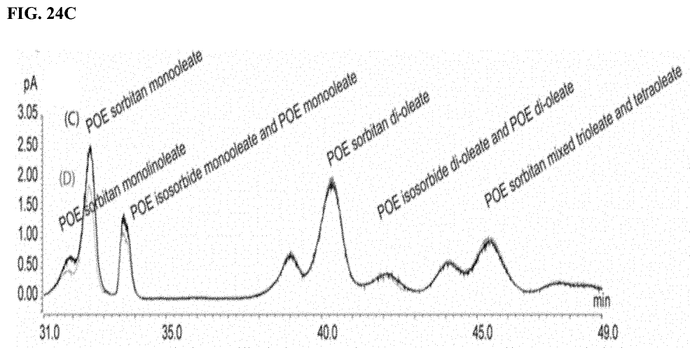

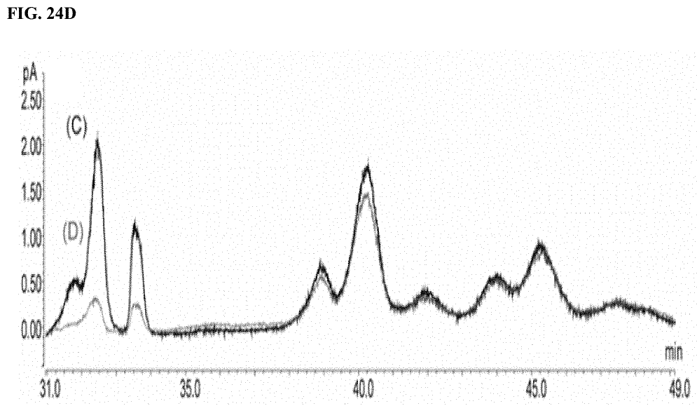

Polysorbates degradation by recombinant human PLBD2 and in-house CHO PLBD2 was monitored as outlined in the Material and Methods section. Recombinant human PLBD2 and in-house CHO PLBD2 with concentration at 200 μg/mL were incubated with 0.1% of PS20 and PS80 for 5 days. Significant PS20 degradation was observed for both human PLBD2 and CHO PLBD2 but in different patterns. By incubating PS20 with OriGene human PLBD2, a decrease in signal intensity occurred on POE-ester peaks eluting at between 27.5 and 38 minutes. The peaks eluted before 34 minutes were POE monoester containing short fatty acid chain, for example, POE sorbitan monolaurate, POE isosorbide monolaurate, POE sorbitan monomyristate and POE isosorbide monomyristate. The POE esters with longer chains, including POE isosorbide monopalmitate, POE isosorbide monosterate and POE sorbitan diester also showed notable reduction (eluted between 34-38 min). Triester and tetraester with higher order esters eluted after 38 minutes, however, did not show noticeable changes during incubation (FIG. 24A). For in-house CHO PLBD2, similar degradation was observed in most PS20 species except the first peak representing POE sorbitan monolaurate (FIG. 24B). As for PS80 degradation, incubation with both human PLBD2 and CHO PLBD2 showed significant degradation on peaks eluting between 30 and 35 minutes, representing POE sorbitan monolinoleate, POE sorbitan monooleate and POE isosorbide monooleate and POE monooleate (FIG. 24C and FIG. 24D). The in-house PLBD2 exhibited higher degree of degradation compared to the commercial one. The in-house PLBD2 also showed a noticeable decrease between elution time of 39 to 41 minutes, representing POE sorbitan dioleate. The distinct degradation pattern induced by these two types of PLBD2 (human vs Chinese hamster) suggested PLBD2 might not be the cause of polysorbate degradation. Instead, because both PLBD2 proteins contain a high level of impurities, it is more likely that the difference in esterase activity originated from some unknown HCP impurities other than from PLBD2 itself.

### Example 15

### Monoclonal Antibody Expressed from PLBD2-Knockout Cell Line Showed No Significant Difference in Lipase Activity Compared to mAb from Control Cell Line with Active PLBD2

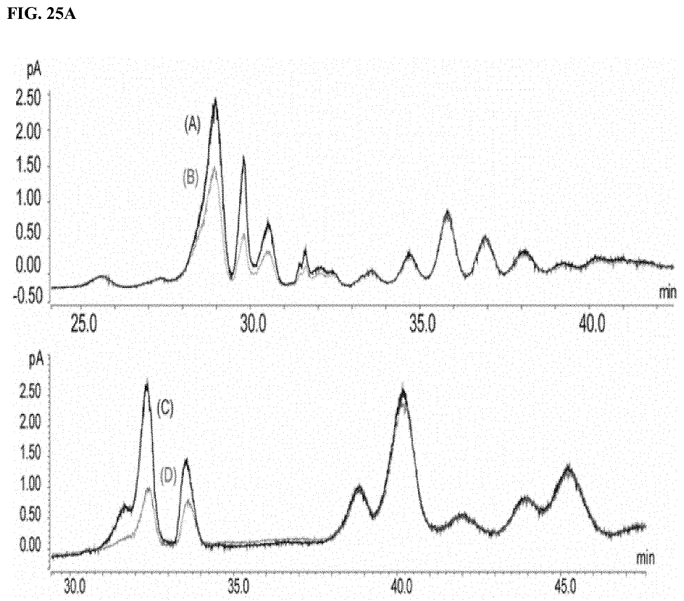

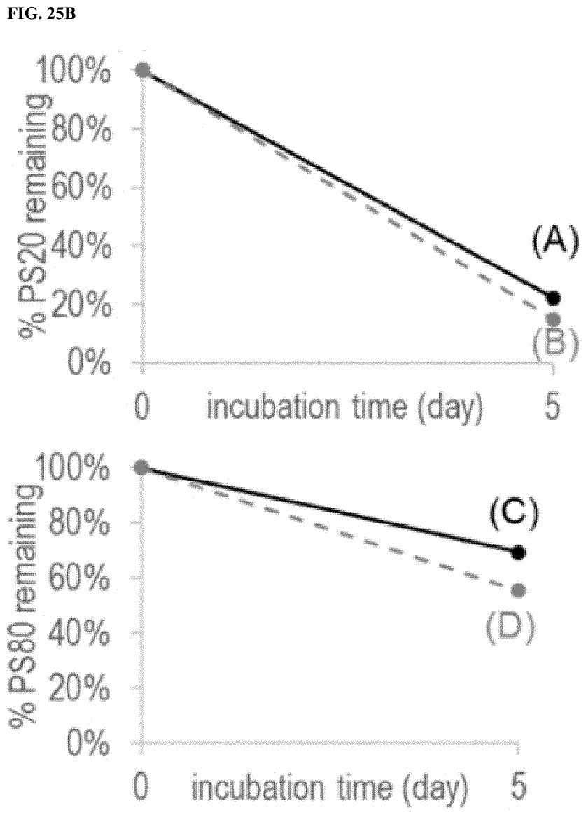

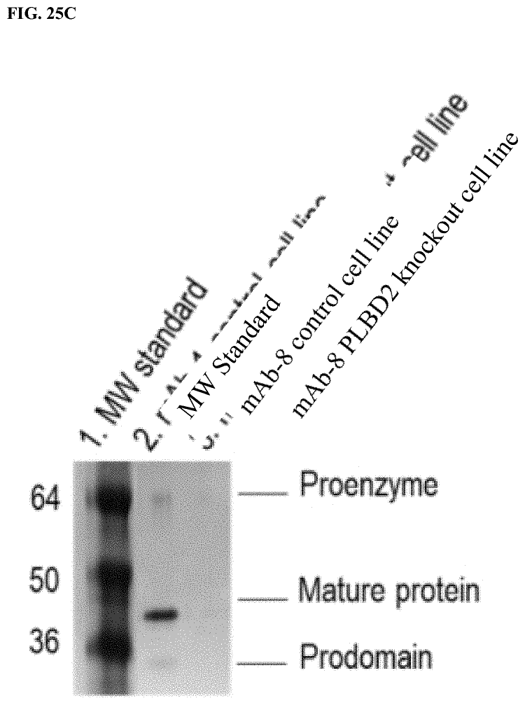

Polysorbate degradation was measured for drug substances produced from either control cell lines or from PLBD2-knockout cell lines before the specific purification that was known to remove PLBD2. Presence of PLBD2 in the control cell line and PLBD2 knockout cell line was determined by western blot analysis. The results clearly showed the clearance of PLBD2 in the knockout cell line (FIG. 25C). The representative degradation profiles of PS20 and PS80 when incubated with mAb-2 (generated by PLBD2 knockout cell line) are demonstrated in FIG. 25A. The percentage of polysorbate degradation is calculated by summing changes in peak areas of monoesters (FIG. 25A). Surprisingly, the lipase activities in antibody expressed from PLBD2 knockout cell line for both PS20 and PS80 were slightly higher than the control cell line (FIG. 25B). If PLBD2 was the cause of PS degradation, it should be able to observe diminished enzymatic activity after the PLBD2 gene was knocked out the PLBD2 does not participate in polysorbate degradation. The PLBD2 knockout cell line generated an alternative active esterase may degrade polysorbates. Proteomics analysis on the mAb-2 (mAb produced by PLBD2 knockout cell line without PLBD2 removal step) was performed, however, no new active lipase was found (data not shown).

### Example 16

### Depletion of PLBD2 does not Result in Decreased Level of PS20 Degradation

To further examine whether PS degradation is caused by the presence of PLBD2 in the formulated mAbs, a PLBD2 depletion experiment was performed. The rationale is that if PS20 degradation is caused by PLBD2, depletion of it will result in a diminished PS20 degradation and the degradation degree will depend on how much PLBD2 has been removed.

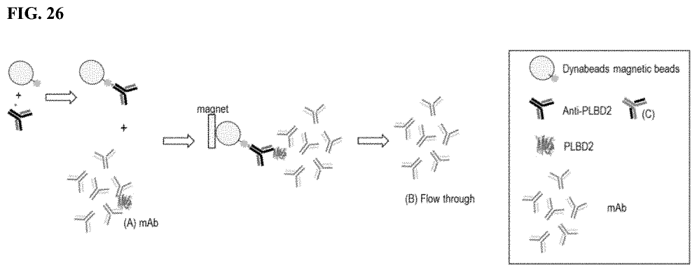

Compared to the knockout experiment, this depletion design will provide clearer results as knockout process may activate new lipases, while depletion will not. FIG. 26 shows the depletion scheme for mAb samples. CHO anti-PLBD2 antibody was covalently coupled to Dynabeads for depletion of PLBD2. It was first validated that anti-PLBD2 was able to bind specifically to PLBD2 by Western blot.

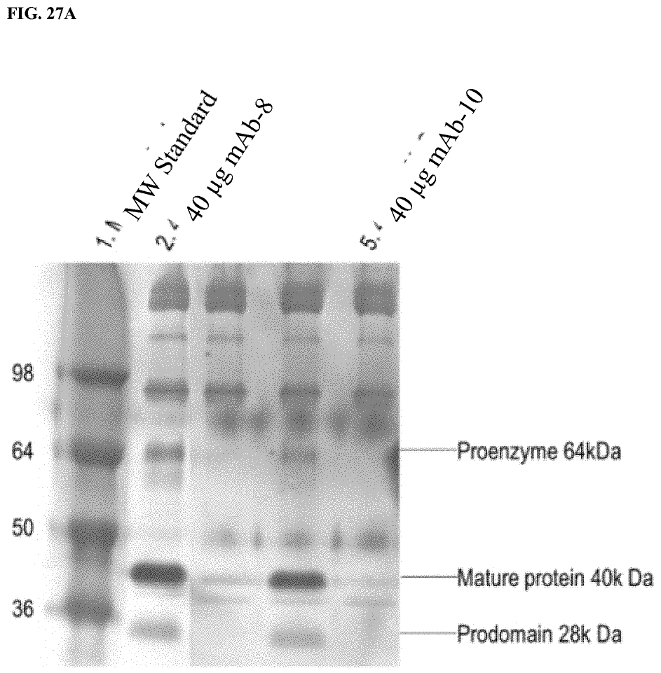

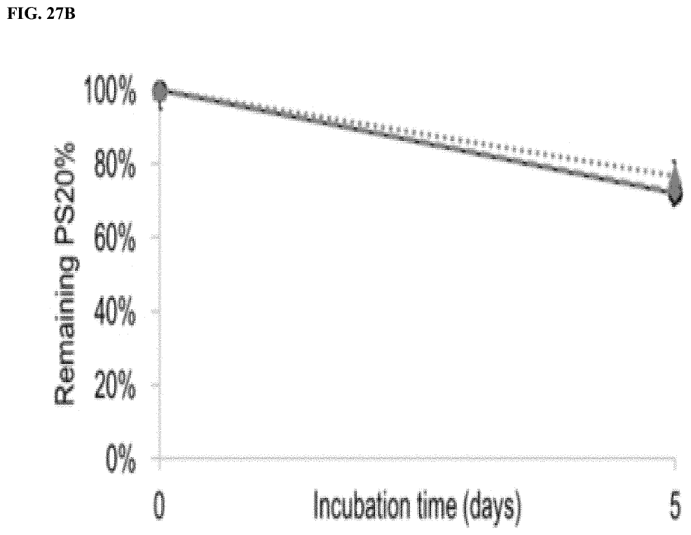

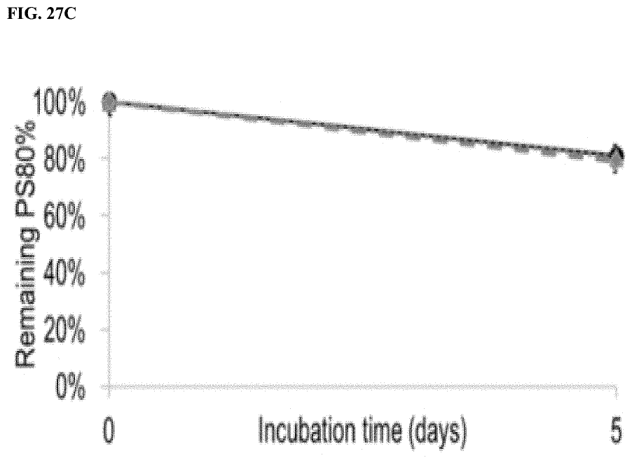

As shown in FIG. 27A, western blot can clearly detect the three forms of PLBD2 present in mAb-8 including proenzyme at 64 kDa, mature protein at 40 k Da and prodomain at 28 kDa (FIG. 27A lane 2). PLBD2 in mAb-8 can be partially (FIG. 27A lane 4) or completely depleted (FIG. 27A lane 3) by adjusting the ratio of anti-PLBD2 and mAb-8 during depletion. Complete depletion of PLBD2 in mAb-8 was performed by incubating 10 mg mAb-8 with 50 μg anti-PLBD2 conjugated magnetic beads while partial depletion of PLBD2 in mAb-8 was achieved by incubating 10 mg mAb-8 with 10 μg anti-PLBD2 conjugated magnetic beads. The percentage of PLBD2 being depleted from mAb-8 was estimated by western blot. Antibody mAb-10 which is PLBD2 free (FIG. 27A Lane 5) served as the negative control.

For mAb-8, before PLBD2-depletion, approximately 28.03% of PS20 degradation was observed at 45° C. for 5 days. After PLBD2-depletion either partially or completely, the esterase activity was measured to be the close to 25%, with 23.21% after partial depletion and 27.68% after complete depletion, respectively (FIG. 27B). Similar results were observed on PS80 degradation (FIG. 27C), with 18.93% PS80 degradation observed in mAb-8, while 19.32% and 20.75% PS80 degradation observed in partially and completely depleted PLBD2 samples, respectively after 5-day incubation at 45° C. The depletion study suggested PLBD2 is clearly not the root cause of polysorbate degradation.

### Example 17

### Amount of PLBD2 in Formulated mAb cannot be Positively Correlated to PS20 Loss Over Time

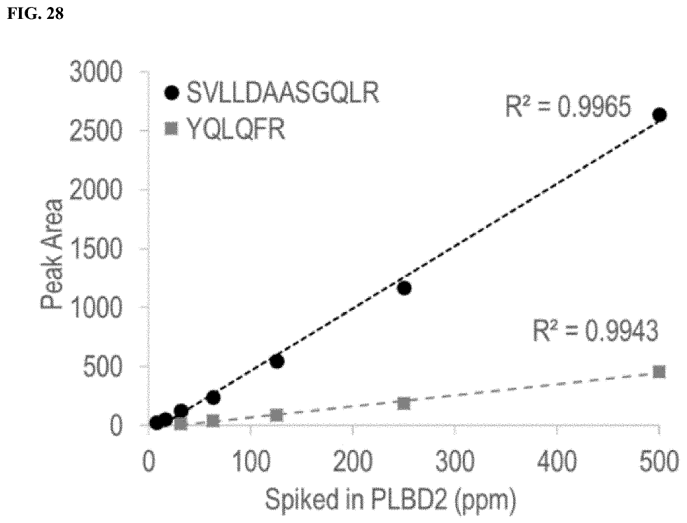

To further prove that PLBD2 is not relevant to polysorbate degradation, PLBD2 quantitation in a number of formulated mAbs was carried out. Two PLBD2 peptides (SVLLDAASGQLR (SEQ ID NO.: 4) and YQLQFR (SEQ ID NO.: 3)) were chosen to quantitate PLBD2 in formulated mAbs using multiple reaction monitoring mass spectrometry (MRM-MS) technology. PLBD2 spiked-in mAb was used to create calibration curve. Standard curves (10-500 ppm) with coefficients 0.9965 and 0.9943 were generated for each of the peptide (FIG. 28), concentration of PLBD2 in each sample was then obtained by extrapolating it peak area onto the curve. In total, 6 mAbs (mAb-10, mAb-11, mAb-12, mAb-13, mAb-14, and mAb-15) were subjected to PLBD2 quantitation. Quantitative examination of peak areas of these 6 mAbs determined the concentration of PLBD2 in the formulated mAb were between 0 to 230 ng/mg mAb.

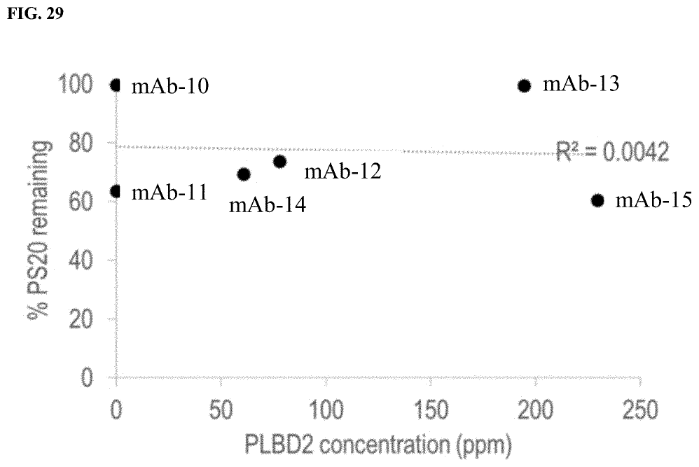

PS20 degradation was measured for the same 6 mAbs after each sample was concentrated and buffer exchanged to 10 mM Histidine buffer, pH 6. The percentage of the remaining PS20 was plotted against PLBD2 concentration and correlation coefficient R² was calculated to evaluate the linear dependence of the two variables (FIG. 29). A slight downhill linear relationship with calculated Pearson correlation coefficient of 0.0042 indicates no correlation between these two variables, suggesting PLBD2 concentration in drug substances is not correlated to the PS20 loss during incubation. Among the samples tested, mAb-11 showed no detectable level of PLBD2 however with strong lipase activity, indicating other lipase/esterase was responsible for PS20 degradation in that drug substance. In contrast, mAb-13 was detected with high concentration of PLBD2, but showed no lipase activity, suggesting PLBD2 is unlikely the root cause of PS20 degradation. The other lipase which is capable of degrading PS20 was detected from mAb-11 and may be the cause for PS20 degradation.

### Example 18

### Impurities Detected and Identified in Commercial PLBD2 and CHO PLBD2

To explain and understand the lipase activity observed when incubating commercial human PLBD2 and in-house CHO PLBD2 with polysorbates, proteomics analysis was conducted to identify potential lipase(s)/esterase present in the commercial human PLBD2 and in-house CHO PLBD2. Results showed that 1,600 host cell proteins were identified in commercial human PLBD2. Among these HCPs, eleven of them were proteins with potential lipase activity as listed in Table 1. One or multiple of those lipases may contribute to polysorbate degradation. For in-house CHO PLBD2, the observed PLBD2 activity most likely resulted from group XV phospholipase A2 (LPLA2), which was the lipase that had been identified to degrade polysorbate by Hall, et. al, as suggested by high confidence identification (16 unique peptides) of this protein in our proteomics analysis (Table 2). LPLA2 was almost exclusively identified, presenting at 0.14% relative to synthetic PLBD2, and showed the exact degradation pattern of PS20 and PS80 as suggested in literature.

TABLE 1
Lipases/esterase identified in commercial human PLBD2

| Protein Name | # Uniq. Peps |
|---|---|
| >sp\|Q8NHP8\|PLBL2_HUMAN Putative Phospholipase B-like 2 | 82 |
| >sp\|Q9NXE4-2\|NSMA3_HUMAN Isoform 2 of Sphingomyelin phosphodiesterase 4 | 12 |
| >sp\|Q8N2K0\|ABD12_HUMAN Monoacylglycerol lipase ABHD12 | 7 |
| >sp\|Q5VWZ2\|LYPL1_HUMAN Lysophospholipase-like protein 1 | 6 |
| >sp\|014734\|ACOT8_HUMAN Acyl-coenzyme A thioesterase 8 | 6 |
| >sp\|Q8NCG7-4\|DGLB_HUMAN Isoform 4 of Sn1-specific diacylglycerol lipase beta | 5 |
| >sp\|Q8IY17\|PLPL6_HUMAN Neuropathy target esterase | 5 |
| >sp\|Q15165\|PON2_HUMAN Serum paraoxonase/arylesterase 2 | 5 |
| >sp\|Q9Y263\|PLAP_HUMAN Phospholipase A-2-activating protein | 4 |
| >sp\|P22413\|ENPP1_HUMAN Ectonucleotide pyrophosphatase/phosphodiesterase 4 | |
| family member 1 | |
| >sp\|Q8IV08\|PLD3_HUMAN Phospholipase D3 | 3 |
| >sp\|Q9BZM\|IPG12A_HUMAN Group XIIA secretory phospholipase A2 | 1 |
| >sp\|1350897\|PPT1_HUMAN Palmitoyl-protein thioesterase 1 | 1 |

TABLE 2
Lipases/esterase identified in CHO PLBD2

| | # Uniq. |
|---|---|
| Protein Name | Peps |
| >tr\|G3I6T1\|G3I6T1_CRIGR Putative Phospholipase B-like 2 | 103 |
| >tr\|G3HKV9\|G3HKV9_CRIGR Group XV phospholipase A2 | 16 |
| >tr\|G3HQY6\|G3HQY6_CRIGR Lipase | 2 |
| >tr\|G3HNQ5\|G3HNQ5_CRIGR Phospholipase D3 | 2 |

Examples 13-18 proved that PLBD2 was not involved in the polysorbate degradation based one three observations—PLBD2-gene knockout did not reduce lipase activity, PLBD2-depleted mAb samples did not show any reduction or elimination of lipase activity, and no positive correlation can be established between PLBD2 concentration and lipase activity. It also showed that the previously identified lipase activity was likely attributable to other lipases that was co-purified together with PLBD2 in the mAb product. These findings resolved the mystery of the lack of correlation between the amount of PLBD2 presented and polysorbate degradation across different companies in the industry, suggesting that the clearance of PLBD2 alone cannot be used as sole indicator for successful purification.

Although PLBD2 had been proved to have no activity on polysorbate degradation and the previous experimental evidence were found coming from the impurities in the synthetic human PLBD2, the work itself shed light on a new direction, which led to the discovery of other problematic host cell proteins.

### Example 19

### LPL Related PS-20 Degradation

Lipoprotein lipase (LPL) has also reported to be associated with PS20 degradation. See Josephine Chiu et al. (supra). In order to study the effect of LPL versus LAL, the amounts of LAL and LPL in mAb-1 (formulated drug product) were studied. For 200 mg/mL mAb-1 incubated with 0.05% PS20, the degradation profile at 0 days, 6 months, 12 months, 18 months, and 24 months was studied on incubation at 5° C. See Table 3. The hydrolysis of PS20 in formulated mAb-1 was examined by mixing 18 μL of mAb-1 (in original formulation or after buffer exchange to 10 mM Histidine, pH 6.0) by collecting aliquot (3 μL) of each solution was diluted twenty-five times by 10 mM Histidine, pH 6 and submitted for LC-CAD analysis as illustrated in Example 1.

TABLE 3
LAL and LPL identified in mAb-1

| Time | Polysorbate concentration (% w/v) | % Recovery |
|---|---|---|
| t = 0 | 0.06 | 100.0 |
| 6 months | 0.06 | 86.0 |
| 12 months | 0.04 | 72.0 |
| 18 months | 0.04 | 66.4 |
| 24 months | 0.03 | 56.8 |

Proteomics analysis was conducted on the sample obtained from the mAb-1 formulation stored for 18 months to identify other potential lipase(s)/esterase present in the mAb-1 formulation. Results are shown in Table 4. Among these HCPs, LAL and LPL are both presented in the mAb-1 formulation in a comparable level.

TABLE 4
Lipases/esterase identified in mAb-1 formulation comprising PS-20 stored at 5° C. for 18
months

| | | | Relative |
|---|---|---|---|
| | Serine Hydrolase/Lipases/ | Number of | Quantitation to |
| | esterase identified | unique peptides | mAb-1 (ppm) |
| G3I6T1 | Putative phospholipase B-like 2 | 6 | 48.28 |
| A0A061I7T0 | Ubiquitin carboxyl-terminal | 5 | 64.58 |
| | hydrolase 42-like protein | | |
| G3IAT2 | Ubiquitin carboxyl-terminal | 2 | 10.4 |
| | hydrolase 31 | | |
| G3HQY6 | Lysosomal acid lipase (LAL) | 2 | 19.99 |
| A0A061IKA1 | Lipoprotein lipase (LPL) | 2 | 15.3 |
| G3H8V5 | Carboxypeptidase | 1 | 0.53 |
| G3GUR1 | Complement C1r-A subcomponent | 1 | 0.32 |

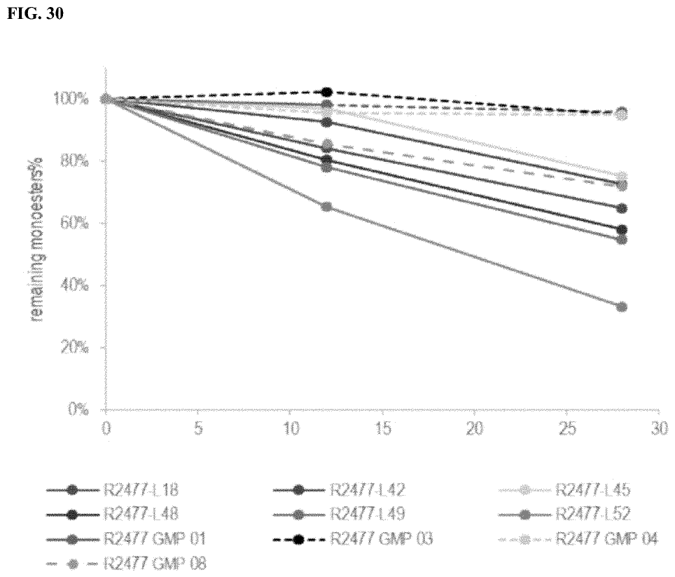

LAL and LPL were also observed in formulations comprising other proteins and PS-20. See Table 5 and FIG. 30. The enrichment for quantification of LAL and LPL was carried our using Proteominer method.

TABLE 5
LAL and LPL identified in other mAbs with lipase acvitiy

| | LPL (ppm) relative to mAb after | LAL (ppm) relative to mAb |
|---|---|---|
| Sample name | enrichment | after enrichment |
| mAb-1 | 15.3 | 19.99 |
| mAb-10 | 20.79 | 16.48 |
| mAb-11 | 15.19 | 15.56 |
| mAb-12 | 20.05 | 24.85 |
| mAb-13 | 1801 | 21.33 |
| mAb-14 | 23.54 | 37.72 |
| mAb-15 | 8.55 | 9.38 |
| mAb-16 | 6.89 | 10.24 |
| mAb-17 | 11.69 | 10.98 |
| mAb-18 | 11.00 | 25.94 |

### Example 20

### PS-20 Degradation Related Better with Concentration of LAL

Samples comprising a 200 mg/mL mAb (mAb-1 and mAb-10-18) were collected in an appendorf tube to which 0.05% PS-20 was added. Each of the tubes were incubated at 37° C. for 28 days. The samples were collected from each tube on day 12 and day 28 for analysis.

Quantification of polysorbate degradation (or remaining PS-20%) in mAb-1 and mAb-10-18 was carried out as outlined in the Material and Methods section. For each of the formulations, the amount of LAL and LPL were measured relative to mAb using the Proteominer method for enrichment.

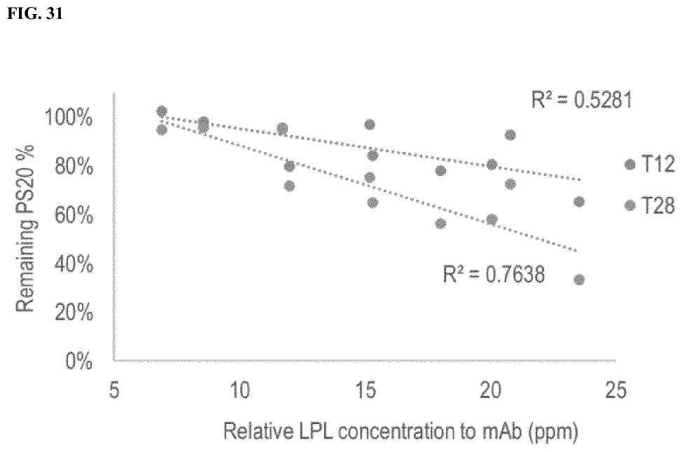

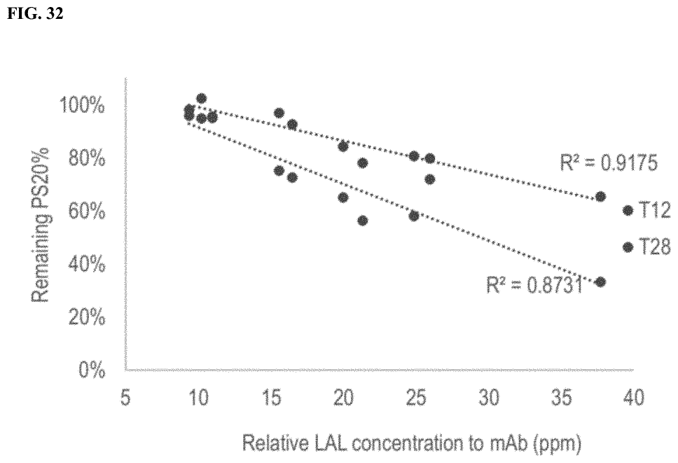

The plot of remaining PS-20% to relative host-cell protein concentration (LAL/LPL) to mAb (ppm) showed a higher correlation with LAL than LPL. See FIGS. 31 and 32. This suggested that LAL causes PS-20 degradation, and not LPL.

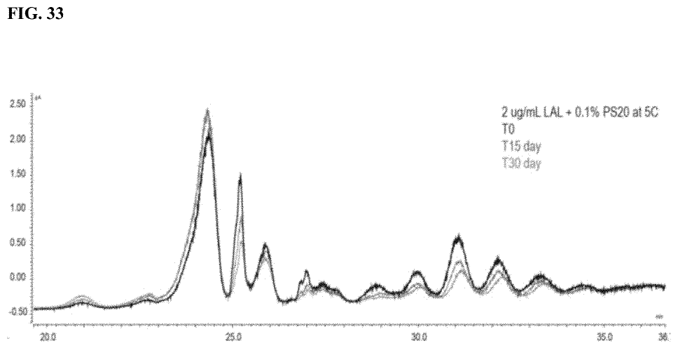

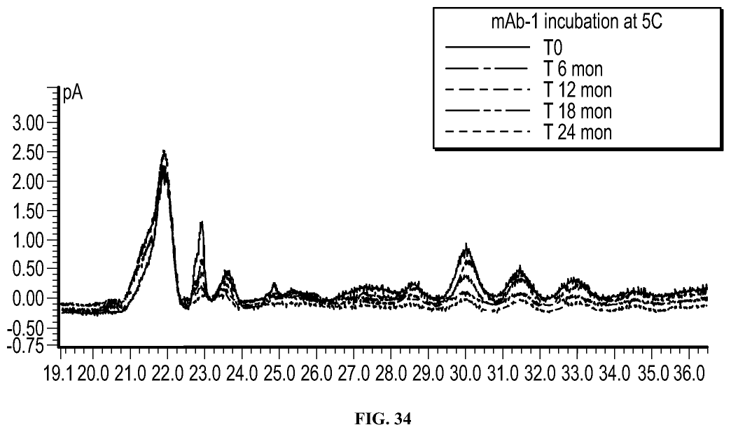

0.1% PS-20 was incubated with 2 μg/mL LAL and stored for 30 days. The incubated sample was analyzed at t=0, 15 days and 30 days. The time course experiment for PS20 degradation in presence of LAL is shown in FIG. 33. The degradation pattern of mAb-1 formulation comprising 0.06% PS-20 was obtained at t=0, 6 months, 12 months, 18 months, and 24 months. See FIG. 34. The two traces of chromatographs are almost identical, suggesting that LAL could be the potential root cause for PS-20 hydrolysis.

### Example 21

### PS-20 Degradation in mAb-1 can be Completely Inhibited by LAL Inhibitor

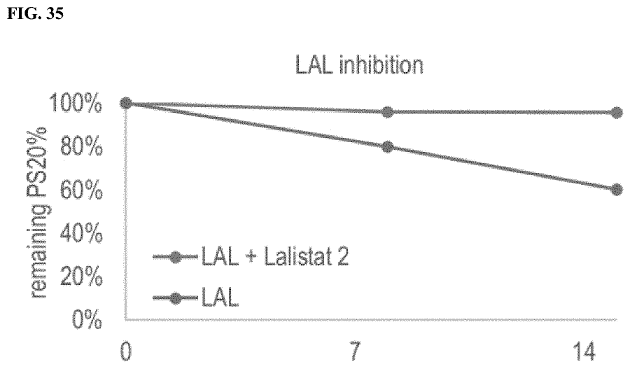

Lalistat 2 is a potent and selective inhibitor of lysosomal acid lipase (LAL) with an IC₅₀ value of 152 nM. FIG. 35 shows a plot of the percentage of PS-20 remaining in samples containing 0.05% PS-20 and 5 μg/mL LAL, with and without Lalistat-2. The incubated samples were analyzed at t=0, 8 days and 15 days. FIG. 35 shows that the sample incubated with Lalistat-2 shows almost no degradation of PS-20.

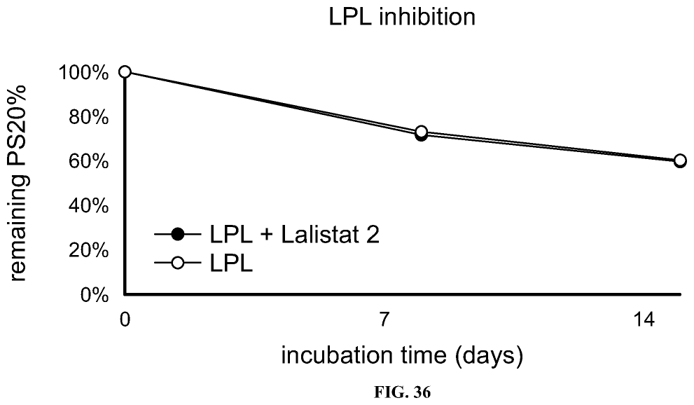

Lalistat 2 does not exhibit inhibition of bovine milk lipoprotein lipase. Thus, addition of Lalistat only causes inhibition of LAL and LPL. FIG. 36 shows a plot of the percentage of PS-20 remaining in samples containing 0.05% PS-20 and 10 μg/mL LPL, with and without Lalistat-2. The incubated samples were analyzed at t=0, 8 days and 15 days. FIG. 36 showed lalistat2 cannot inhibit LPL's activity. Therefore, if lalistat2 can inhibit lipase activity in mAbs completely, then it means the LPL detected from mAbs were not active or abundance are too low to degrade PS20. The PS20 degradation seen from recombinant LPL may either due to the only LPL in high concentration can degrade PS20, or due to unexpected impurities presented in the recombinant LPL.

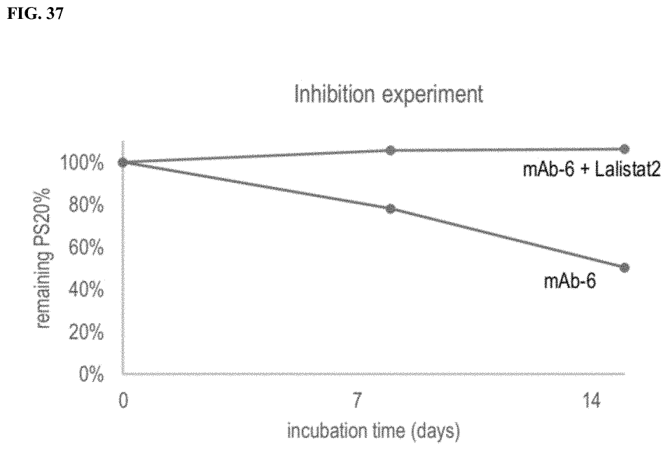

mAb-15 comprising 0.05% PS-20 was incubated at 37° C. for 15 days in presence and absence of 0.1 M Lalistat-2. The incubated samples were analyzed at t=0, 8 days and 14 days. FIG. 37 shows the percentage of PS-20 remaining in mAb-15 formulations incubated with and without Lalistat. The sample incubated with Lalistat-2 shows almost no degradation of PS-20. Thus, despite the presence of LPL in the mAb-6 formulation (see Table 5), PS-20 did not degrade showing that LAL and not LPL is responsible of PS-20 degradation in protein formulations.

What is claimed is:

1. A method of preparing a composition having a protein of interest, comprising
culturing mammalian cells to obtain a sample matrix having protein of interest and lysosomal acid lipase;
contacting the sample matrix to a first chromatography resin;
washing the bound protein of interest to form an eluate;
contacting the eluate to a second chromatography resin;
collecting a flow-through from washing the second chromatography resin;
contacting the flow-through to a third chromatography resin;
collecting a second flow-through from washing the third chromatography resin; and
filtering the second flow-through by viral filtration to obtain the composition, wherein the composition has less than about 1 ppm of lysosomal acid lipase.

2. The method of claim 1, wherein the lysosomal acid lipase in the sample matrix is more than about 15 ppm.

3. The method of claim 1, wherein the mammalian cells are CHO cells.

4. The method of claim 1, wherein the first chromatographic resin is protein A chromatographic resin.

5. The method of claim 1, wherein the second chromatographic resin is anion-exchange chromatographic resin.

6. The method of claim 1, wherein the third chromatographic resin is hydrophobic interaction chromatographic resin.

7. The method of claim 1 further comprising contacting the eluate to beads having anti-lysosomal acid lipase antibody.

8. The method of claim 1 further comprising contacting the flow-through to beads having anti-lysosomal acid lipase antibody.

9. The method of claim 1 further comprising contacting the second flow-through to beads having anti-lysosomal acid lipase antibody.

10. A method of depleting lysosomal acid lipase levels in a sample matrix, comprising
contacting the sample matrix having lysosomal acid lipase to a resin having anti-lysosomal acid lipase antibody;
washing the resin with a wash buffer; and
collecting wash fractions from the washing, wherein the wash fractions have a reduced concentration of lysosomal acid lipase than lysosomal acid lipase in sample matrix.

11. The method of claim 10, wherein the lysosomal acid lipase in the sample matrix is more than about 15 ppm.

12. The method of claim 10, wherein the sample matrix comprises polysorbate.

13. The method of claim 10, wherein the resin is a magnetic bead.

14. The method of claim 10, wherein the amount of anti-lysosomal acid lipase antibody to the resin is about 1 μg/g to about 50 μg/g.

15. The method of claim 10, wherein the anti-lysosomal acid lipase antibody is of human origin.

16. The method of claim 10, wherein the anti-lysosomal acid lipase antibody is of hamster origin.

17. The method of claim 10, wherein the wash fractions have a have a two-fold reduced amount of lysosomal acid lipase compared to amount of lysosomal acid lipase in the sample matrix.

18. A method of detecting lysosomal acid lipase in a sample matrix, comprising:
contacting the sample matrix with a biotinylated anti-lysosomal acid lipase antibody;
incubating the sample matrix with a resin;
performing elution on the resin of to form an eluate;
adding hydrolyzing agent to the eluate to obtain digests; and
analyzing the digests to detect the lysosomal acid lipase.

19. The method of claim 18, wherein the resin is a magnetic bead.

20. The method of claim 18, wherein the digests are analyzed using a mass spectrometer coupled to a liquid chromatography-multiple reaction monitoring system.


SEQUENCE LISTING (13 sequences)

SEQ ID NO: 1 - protein, 9 aa, Artificial Sequence
  Description of Artificial Sequence Synthetic peptide
LLSLTYDQK

SEQ ID NO: 2 - protein, 12 aa, Artificial Sequence
  Description of Artificial Sequence Synthetic peptide
ELAVAAAYQSVR

SEQ ID NO: 3 - protein, 6 aa, Artificial Sequence
  Description of Artificial Sequence Synthetic peptide
YQLQFR

SEQ ID NO: 4 - protein, 12 aa, Artificial Sequence
  Description of Artificial Sequence Synthetic peptide
SVLLDAASGQLR

SEQ ID NO: 5 - DNA, 20 bp, Artificial Sequence
  Description of Artificial Sequence Synthetic oligonucleotide
actgcaggtatgtgagtgct

SEQ ID NO: 6 - DNA, 20 bp, Artificial Sequence
  Description of Artificial Sequence Synthetic oligonucleotide
ggattacgaatgtcaccctg

SEQ ID NO: 7 - DNA, 20 bp, Artificial Sequence
  Description of Artificial Sequence Synthetic oligonucleotide
ttggggaggtaagtgtacgt

SEQ ID NO: 8 - DNA, 20 bp, Artificial Sequence
  Description of Artificial Sequence Synthetic oligonucleotide
gtactggggatacccgagtg

SEQ ID NO: 9 - DNA, 20 bp, Artificial Sequence
  Description of Artificial Sequence Synthetic oligonucleotide
ccagttgtctatcttcagca

SEQ ID NO: 10 - DNA, 33 bp, Artificial Sequence
  Description of Artificial Sequence Synthetic oligonucleotide
tgtatgagaccacgcccccatggaccggagccc

SEQ ID NO: 11 - DNA, 32 bp, Artificial Sequence
  Description of Artificial Sequence Synthetic oligonucleotide
aaacgggctccggtccatggggcgtggtctca

SEQ ID NO: 12 - protein, 550 aa, Cricetulus griseus
MVSLGSVFGLLLLLIIVRASRSAGIDFRFGSYIDNYMVLQKEPSGAVIWGFGTPGATVTVTLCQGQNTFKKKVTTVKAHSNTWVVVLDPMKPGGPYEVMAKQTFGITNVTLRIHDILFGDVWLCSGQSNMKMTVLQIFNASKELAVAAAYQSVRIFSASLVQAEEELEDLDKVDLSWSRPTAGNLGNGNYTYMSAVCWLFGRYLYDNLQYPIGLVSSSWGGTPIEAWSSKRALKSCGVPNTSNERILQTTRSECKSEGSLCPLGSFTLPPSVTGPATHSVLWNAMIHPLQKMTLKGVIWYQGESNEDLNRDLYSCMFPALIDDWRQTFHDGSQKQTERFFPFGFVQLSSYAETNTSDYRFPEIRWHQTADFGFVPNLKMPNTFMAVAMDLCDLNSPFGSIHPRDKQTVAYRLHLGARALAYGEKNLAFQGPLPKKIELLAHSRLLSLTYDQKIQVHRQDNKSFEISCCSDRLCKWLPVSVNSFSTKTLILGISACRGTVVDVRYAWTTWPCDYKQCAVYHPSNTLPAPPFTAPITYQAPRFRASDSSTQE

SEQ ID NO: 13 - protein, 523 aa, Homo sapiens
MVAPGLVLGLVLPLILWADRSAGIGFRFASYINNDMVLQKEPAGAVIWGFGTPGATVTVTLRQGQETIMKKVTSVKAHSDTWMVVLDPMKPGGPFEVMAQQTLEKINFTLRVHDVLFGDVWLCSGQSNMQMTVLQIFNATRELSNTAAYQSVRILSVSPIQAEQELEDLVAVDLQWSKPTSENLGHGYFKYMSAVCWLFGRHLYDTLQYPIGLIASSWGGTPIEAWSSGRSLKACGVPKQGSIPYDSVTGPSKHSVLWNAMIHPLCNMTLKGVVWYQGESNINYNTDLYNCTFPALIEDWRETFHRGSQGQTERFFPFGLVQLSSDLSKKSSDDGFPQIRWHQTADFGYVPNPKMPNTFMAVAMDLCDRDSPFGSIHPRDKQTVAYRLHLGARALAYGEKNLTFEGPLPEKIELLAHKGLLNLTYYQQIQVQKKDNKIFEISCCSDHRCKWLPASMNTVSTQSLTLAIDSCHGTVVALRYAWTTWPCEYKQCPLYHPSSALPAPPFIAFITDQGPGHQSNVAK